(19) United States
(12) Patent Application Publication
(10) Pub. No.: US 2010/0144798 A1
(43) Pub. Date: Jun. 10, 2010

(54) Modulators of ATP-Binding Cassette Transporters

(75) Inventors: Frederick F. VanGoor, San Diego, CA (US); Sara Sabina Hadida Ruah, La Jolla, CA (US); Ashvani Kumar Singh, San Diego, CA (US); Eric R. Olson, Belmont, MA (US); Lewis Ray Makings, Encinitas, CA (US); Jesus E. Gonzalez, III, San Diego, CA (US); James Arvid Rader, La Jolla, CA (US); Fred Chambers, III, San Diego, CA (US); Mark Thomas Miller, San Diego, CA (US); Peter Diederik Jan Grootenhuis, San Diego, CA (US); Yahua Liu, San Diego, CA (US)

Correspondence Address: VERTEX PHARMACEUTICALS INC., 130 WAVERLY STREET, CAMBRIDGE, MA, 02139-4242, US

(73) Assignee: VERTEX PHARMACEUTICALS INCORPORATED, Cambridge, MA (US)

(21) Appl. No.: 12/544,323

(22) Filed: Aug. 20, 2009

Related U.S. Application Data
(62) Division of application No. 10/800,022, filed on Mar. 12, 2004, now abandoned.
(60) Provisional application No. 60/453,978, filed on Mar. 12, 2003.

(51) Int. Cl. A61K 31/4439; A61K 31/415; A61P 1/12; A61P 13/12; A61P 15/10; A61P 27/02; C12N 5/00

(52) U.S. Cl. 514/341; 514/406; 435/375

(57) ABSTRACT

The present invention relates to modulators of ATP-Binding Cassette (“ABC”) transporters or fragments thereof, including Cystic Fibrosis Transmembrane Regulator (“CFTR”), compositions thereof, and methods therewith. The present invention also relates to methods of treating ABC transporter mediated diseases using such modulators.

### CROSS-REFERENCE TO RELATED APPLICATIONS

The present application is a division of U.S. patent application Ser. No. 10/800,022 filed Mar. 12, 2004 and entitled “MODULATORS OF ATP-BINDING CASSETTE TRANSPORTERS,” which claims the benefit under 35 U.S.C. §119 of U.S. Provisional Application No. 60/453,978, filed Mar. 12, 2003, the entire contents of each of the above applications being incorporated herein by reference.

### TECHNICAL FIELD OF THE INVENTION

The present invention relates to modulators of ATP-Binding Cassette (“ABC”) transporters or fragments thereof, including Cystic Fibrosis Transmembrane Regulator (“CFTR”), compositions thereof, and methods therewith. The present invention also relates to methods of treating ABC transporter mediated diseases using such modulators.

### BACKGROUND OF THE INVENTION

ABC transporters are a family of membrane transporter proteins that regulate the transport of a wide variety of pharmacological agents, potentially toxic drugs, and xenobiotics, as well as anions. ABC transporters are homologous membrane proteins that bind and use cellular adenosine triphosphate (ATP) for their specific activities. Some of these transporters were discovered as multidrug resistance proteins (like the MDR1-P glycoprotein, or the multidrug resistance protein, MRP1), defending malignant cancer cells against chemotherapeutic agents. To date, 48 ABC Transporters have been identified and grouped into 7 families based on their sequence identity and function.

ABC transporters regulate a variety of important physiological roles within the body and provide defense against harmful environmental compounds. Because of this, they represent important potential drug targets for the treatment of diseases associated with defects in the transporter, prevention of drug transport out of the target cell, and intervention in other diseases in which modulation of ABC transporter activity may be beneficial.

One member of the ABC transporter family commonly associated with disease is the cAMP/ATP-mediated anion channel, CFTR. CFTR is expressed in a variety of cells types, including absorptive and secratory epithelia cells, where it regulates anion flux across the membrane, as well as the activity of other ion channels and proteins. In epithelia cells, normal functioning of CFTR is critical for the maintenance of electrolyte transport throughout the body, including respiratory and digestive tissue. CFTR is composed of approximately 1480 amino acids that encode a protein made up of a tandem repeate of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). A defect in this gene causes mutations in CFTR resulting in Cystic Fibrosis (“CF”), the most common fatal genetic disease in humans. Cystic Fibrosis affects approximately one in every 2,500 infants in the United States. Within the general United States population, up to 10 million people carry a single copy of the defective gene without apparent ill effects. In contrast, individuals with two copies of the CF associated gene suffer from the debilitating and fatal effects of CF, including chronic lung disease.

In patients with cystic fibrosis, mutations in CFTR endogenously expressed in respiratory epithelia leads to reduced apical anion secretion causing an imbalance in ion and fluid transport. The resulting decrease in anion transport contributes to enhanced mucus accumulation in the lung and the accompanying microbial infections that ultimately cause death in CF patients. In addition to respiratory disease, CF patients typically suffer from gastrointestinal problems and pancreatic insufficiency that, if left untreated, results in death. In addition, the majority of Males with cystic fibrosis are infertile and fertility is decreased among females with cystic fibrosis. In contrast to the severe effects of two copies of the CF associated gene, individuals with a single copy of the CF associated gene exhibit increased resistance to cholera and to dehydration resulting from diarrhea—perhaps explaining the relatively high frequency of the CF gene within the population.

Sequence analysis of the CFTR gene of CF chromosomes has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, >1000 disease causing mutations in the CF gene have been identified (http://www.genet.sickkids.on.ca/cftr/). The most prevalent mutation is a deletion of phenylalanine at position 508 of the CFTR amino acid sequence, and is commonly referred to as ΔF508-CFTR. This mutation occurs in approximately 70% of the cases of cystic fibrosis and is associated with a severe disease.

The deletion of residue 508 in ΔF508-CFTR prevents the nascent protein from folding correctly. This results in the inability of the mutant protein to exit the ER, and traffic to the plasma membrane. As a result, the number of channels present in the membrane is far less than observed in cells expressing wild-type CFTR. In addition to impaired trafficking, the mutation results in defective channel gating. Together, the reduced number of channels in the membrane and the defective gating lead to reduced anion transport across epithelia leading to defective ion and fluid transport. (Quinton, P. M. (1990), FASEB J. 4: 2709-2727). Studies have shown, however, that the reduced numbers of ΔF508-CFTR in the membrane are functional, albeit less than wild-type CFTR. (Dalemans et al. (1991), Nature Lond. 354: 526-528; Denning et al., supra.; Pasyk and Foskett (1995), J. Cell. Biochem. 270: 12347-50). In addition to ΔF508-CFTR, other disease causing mutations in CFTR that result in defective trafficking, synthesis, and/or channel gating could be up- or down-regulated to alter anion secretion and modify disease progression and/or severity.

Although CFTR transports a variety of molecules in addition to anions, it is clear that this role (the transport of anions) represents one element in an important mechanism of transporting ions and water across the epithelium. The other elements include the epithelial Na⁺ channel, ENaC, Na⁺/2Cl⁻/K⁺ co-transporter, Na⁺—K⁺-ATPase pump and the basolateral membrane channels, that are responsible for the uptake of chloride into the cell.

These elements work together to achieve directional transport across the epithelium via their selective expression and localization within the cell. Chloride absorption takes place by the coordinated activity of ENaC and CFTR present on the apical membrane and the Na⁺—K⁺-ATPase pump and Cl− channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

In addition to Cystic Fibrosis, modulation of CFTR activity may be beneficial for other diseases not directly caused by mutations in CFTR. These include, but are not limited to, chronic obstructive pulmonary disease (COPD), dry eye disease, and Sjögren's Syndrome.

COPD is characterized by airflow limitation that is progressive and not fully reversible. The airflow limitation is due to mucus hypersecretion, emphysema, and bronchiolitis. Activators of mutant or wild-type CFTR offer a potential treatment of mucus hypersecretion and impaired mucociliary clearance that is common in COPD. Specifically, increasing anion secretion across CFTR may facilitate fluid transport into the airway surface liquid to hydrate the mucus and optimized periciliary fluid viscosity. This would lead to enhanced mcuociliary clearance and a reduction in the symptoms associated with COPD.

Dry eye disease is characterized by a decrease in tear aqueous production and abnormal tear film lipid, protein and mucin profiles. There are many causes of dry eye, some of which include age, Lasik eye surgery, arthritis, medications, chemical/thermal burns, allergies, and diseases, such as Cystic Fibrosis and Sjögrens's syndrome. Increasing anion secretion via CFTR would enhance fluid transport from the corneal endothelial cells and secretory glands surrounding the eye to increase corneal hydration. This would help to alleviate the symptoms associated with dry eye disease.

Sjögrens's syndrome is an autoimmune disease in which the immune system attacks moisture-producing glands throughout the body, including the eye, mouth, skin, respiratory tissue, liver, vagina, and gut. Symptoms, include, dry eye, mouth, and vagina, as well as lung disease. The disease is also associated with rheumatoid arthritis, systemic lupus, systemic sclerosis, and polymypositis/dermatomyositis. Defective protein trafficking is believed to cause the disease, for which treatment options are limited. Modulators of CFTR activity may hydrate the various organs afflicted by the disease and help to elevate the associated symptoms.

Compounds that are found to modulate CFTR activity by modulating protein folding may be beneficial for the treatment of a wide variety of other protein folding diseases, including, but not limited to, cancer (due to mutations in the tumor suppressor protein, p53), Prion disease, α-1 antitrypsin deficiency, hereditary nephrogenic diabetes insipidus, and Dubin Johnson Syndrome.

In addition to up-regulation of CFTR activity, reducing anion secretion by CFTR modulators may be beneficial for the treatment of secretory diarrheas, in which epithelial water transport is dramatically increased as a result of secretagogue activated chloride transport. The mechanism involves elevation of cAMP and stimulation of CFTR.

Although there are numerous causes of diarrhea, the major consequences of diarrheal diseases, resulting from excessive chloride transport are common to all, and include dehydration, acidosis, death and impaired growth.

Acute and chronic diarrheas represent a major medical problem in many areas of the world. Diarrhea is both a significant factor in malnutrition and the leading cause of death (5,000,000 deaths/year) in children less than five years old.

Secretory diarrheas are also a dangerous condition in patients of acquired immunodeficiency syndrome (AIDS) and chronic inflammatory bowel disease (IBD). 16 million travelers to developing countries from industrialized nations every year develop diarrhea, with the severity and number of cases of diarrhea varying depending on the country and area of travel.

Diarrhea in barn animals and pets such as cows, pigs and horses, sheep, goats, cats and dogs, also known as scours, is a major cause of death in these animals. Diarrhea can result from any major transition, such as weaning or physical movement, as well as in response to a variety of bacterial or viral infections and generally occurs within the first few hours of the animal's life.

The most common diarrheal causing bacteria is enterotoxogenic E-coli (ETEC) having the K99 pilus antigen. Common viral causes of diarrhea include rotavirus and coronavirus. Other infectious agents include cryptosporidium, giardia lamblia, and salmonella, among others.

Symptoms of rotaviral infection include excretion of watery feces, dehydration and weakness. Coronavirus causes a more severe illness in the newborn animals, and has a higher mortality rate than rotaviral infection. Often, however, a young animal may be infected with more than one virus or with a combination of viral and bacterial microorganisms at one time. This dramatically increases the severity of the disease.

Accordingly, there is a need for modulators of an ABC transporter activity, and compositions thereof, that can be used to modulate the activity of the ABC transporter in the cell membrane of a mammal.

There is a need for methods of treating ABC transporter mediated diseases using such modulators of ABC transporter activity.

There is a need for methods of modulating an ABC transporter activity in an ex vivo cell membrane of a mammal.

There is a need for modulators of CFTR activity that can be used to modulate the activity of CFTR in the cell membrane of a mammal.

There is a need for methods of treating CFTR-mediated diseases using such modulators of CFTR activity.

There is a need for methods of modulating CFTR activity in an ex vivo cell membrane of a mammal.

### SUMMARY OF THE INVENTION

The present invention provides a method of modulating ABC transporter activity, comprising the step of contacting said ABC transporter with a compound of formula (I):

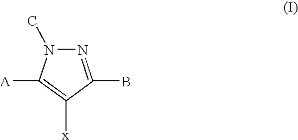

or a pharmaceutically acceptable salt thereof; wherein:

A and B are independently selected from aryl, heterocyclic, heteroaryl, or cycloaliphatic ring;

C is H, aryl, heterocyclic, heteroaryl, cycloaliphatic, aliphatic, C(O)R², C(O)R³, C(O)NH₂, C(O)NHR², C(O)NHR³, C(O)N(R²)₂, C(O)N(R³)₂;

X is H, (CH₂)ₙ—Y, R², R³, R⁴, R⁵, or R⁶;

wherein each of A, B, C, and X optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶ or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R²_{, R}⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CHF₂, CH₂F, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group.

The present invention also provides compositions comprising compounds of formula (I), and methods of treating ABC transporter mediated diseases using compounds of formula (I).

### DETAILED DESCRIPTION OF THE INVENTION

As used herein, the following definitions shall apply unless otherwise indicated.

The term “ABC-transporter” as used herein means an ABC-transporter protein or a fragment thereof comprising at least one binding domain, wherein said protein or fragment thereof is present in vivo or in vitro. The term “binding domain” as used herein means a domain on the ABC-transporter that can bind to a modulator. See, e.g., Hwang, T. C. et al., J. Gen. Physiol. (1998): 111(3), 477-90. CFTR is an example of an ABC-transporter.

The term “CFTR” as used herein means cystic fibrosis transmembrane regulator or a mutation thereof capable of regulator activity, including, but not limited to, ΔF508 CFTR and G551D CFTR (see, e.g., http://www.genet.sickkids.on.ca/cftr/, for CFTR mutations).

The term “modulating” as used herein means increasing or decreasing by a measurable amount. Suitable means for such measurements are well known in the art.

The phrase “optionally substituted” is used interchangeably with the phrase “substituted or unsubstituted.”

The term “aliphatic” or “aliphatic group”, as used herein, means a straight-chain or branched, substituted or unsubstituted hydrocarbon chain that is completely saturated (alkyl) or is unsaturated (alkenyl or alkynyl). Unless otherwise specified, an aliphatic group has 1 to 12 carbon atoms, preferably, 1-6 carbon atoms, and more preferably, 1-4 carbon atoms. Unless otherwise specified, up to three, and preferably up to two, —CH₂— in said aliphatic may be replaced with O, S, or —NH.

The term “cycloaliphatic” means a saturated or partically unsaturated monocyclic or bicyclic hydrocarbon ring that has a single point of attachment to the rest of the molecule. Unless otherwise specified, preferred cycloaliphatic rings are 3-8 membered monocyclic rings, more preferably 3-6, and ever more preferably, 3, 5, or 6. Also preferred, unless otherwise specified, are 8-12 membered bicyclic rings, more preferably 10 membered bicyclic rings.

The term “heteroatom,” unless otherwise specified, means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or as in N-substituted pyrrolidinyl.

The term “unsaturated”, as used herein, means a double bond or a triple bond. Each such bond constitutes one unit of unsaturation.

The term “aryl” used alone or as part of a larger moiety as in “aralkyl”, “aralkoxy”, or “aryloxyalkyl”, refers to monocyclic, bicyclic and tricyclic aromatic carbocyclic ring systems. Unless otherwise specified, preferred aryl rings have a total of five to fourteen ring members, wherein at least one ring, if bicyclic or tricyclic, in the system is aromatic and wherein each ring in the system contains up to 6 ring members. The term “aryl” may be used interchangeably with the term “aryl ring”. Phenyl is an example of aryl.

The term “heterocycle”, “heterocyclyl”, “heterocycloaliphatic”, or “heterocyclic” as used herein means non-aromatic, monocyclic, bicyclic or tricyclic ring systems, wherein one or more ring members is a heteroatom. Unless otherwise specified, each ring in the system preferably contains 3 to 7 ring members with preferably 1-3 heteroatoms.

The term “heteroaryl”, used alone or as part of a larger moiety as in “heteroaralkyl” or “heteroarylalkoxy”, refers to monocyclic, bicyclic and tricyclic ring systems, wherein at least one ring in the system is aromatic, and at least one ring in the system contains one or more heteroatoms. Unless otherwise specified, such ring systems preferably have a total of 5 to 15 ring members, wherein each ring in the system preferably contains 3 to 7 ring members, with preferably 1-3 heteroatoms. The term “heteroaryl” may be used interchangeably with the term “heteroaryl ring” or the term “heteroaromatic”.

A combination of substituents or variables is permissible only if such a combination results in a stable or chemically feasible compound. A stable compound or chemically feasible compound is one that is not substantially altered when kept at a temperature of 40° C. or less, in the absence of moisture or other chemically reactive conditions, for at least a week.

The present invention provides a method of modulating ABC transporter activity, comprising the step of contacting said ABC transporter with a compound of formula (I):

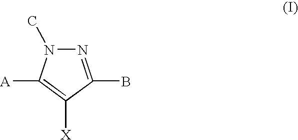

or a pharmaceutically acceptable salt thereof;
wherein:

A and B are independently selected from aryl, heterocyclic, heteroaryl, or cycloaliphatic ring;

C is H, aryl, heterocyclic, heteroaryl, cycloaliphatic, aliphatic, C(O)R², C(O)R³, C(O)NH₂, C(O)NHR², C(O)NHR³, C(O)N(R²)₂, C(O)N(R³)₂;

X is H, (CH₂)ₙ—Y, R², R³, R⁴, R⁵, or R⁶;

wherein each of A, B, C, and X optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶ or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R²_{, R}⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CHF₂, CH₂F, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic), or O-aliphatic; and

R⁸ is an amino protecting group.

The term “amino protecting group” refers to a suitable chemical group that may be attached to a nitrogen atom. The term “protected” refers to when the designated functional group is attached to a suitable chemical group (protecting group). Examples of suitable amino protecting groups and protecting groups are described in T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); L. Paquette, ed. Encyclopedia of Reagents for Organic Synthesis, John Wiley and Sons (1995), the disclosures whereof is incorporated herein by reference.

According to one embodiment, C is H. Or, C is C(O)CH₃, C(O)Ph, phenyl, C(O)NH(C1-C4)-alkyl, or C(O)N[(C1-C4)-alkyl]₂.

According to another embodiment, C is optionally substituted H, aryl, heterocyclic, heteroaryl, cycloaliphatic, aliphatic.

According to one embodiment, X is H. Or, X is X is (CH₂)ₙ—Y. According to another embodiment, X is R². Or, X is R³. According to yet another embodiment, X is R⁴.

According to one embodiment, A and B are independently selected from optionally substituted C6-C10 aryl. Or, A is an optionally substituted phenyl.

According to one embodiment, A and B are independently selected from optionally substituted C5-C10 heteroaryl. Or, A and B each is an optionally substituted C5-C7 heteroaryl.

According to another embodiment, A and B are independently selected from phenyl, triazinyl, pyrazinyl, pyrimidinyl, pyridazinyl, pyridinyl, thiadiazolyl, triazolyl, oxadiazolyl, isothiazolyl, pyrazolyl, imidazolyl, thiazolyl, oxazolyl, pyrrolyl, thiophenyl, furanyl, indolizinyl, indolyl, isoindolyl, benzofuranyl, benzo[b]thiophenyl, 1H-indazolyl, benzimidazolyl, benzthiazolyl, purinyl, quinolinyl, isoquinolinyl, cinnolinyl, phthazinyl, quinazolinyl, quinoxalinyl, 1,8-naphthyridinyl, pteridinyl, carbazolyl, acridinyl, phenazinyl, phenothiazinyl, phenoxazinyl, indenyl, naphthyl, azulinyl, or anthracenyl.

According to another embodiment, A and B are independently selected from optionally substituted phenyl, pyrazolyl, pyridyl, thiazolyl, oxazolyl, thiophenyl, or furanyl. Or, A and B are independently selected from phenyl, furanyl, or pyridyl.

According to another embodiment, A and B are independently selected from optionally substituted phenyl, pyridyl, thiophenyl, or furanyl.

According to another embodiment, A is optionally substituted phenyl. Exemplary embodiments of A include 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl, 2-hydroxy-5-methylsulfonylphenyl, 2-hydroxy-5-amidophenyl, 2-hydroxy-6-methoxyphenyl, 2-hydroxy-4,6-dimethylphenyl, 2-hydroxy-4,5-dimethylphenyl, 2-hydroxy-4-methylphenyl, or 2-hydroxy-4-fluorophenyl.

Preferred embodiments of B include the following optionally substituted ring systems:

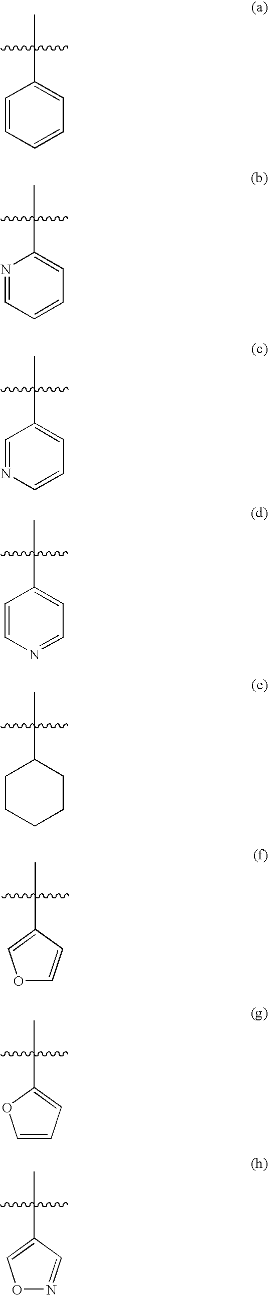

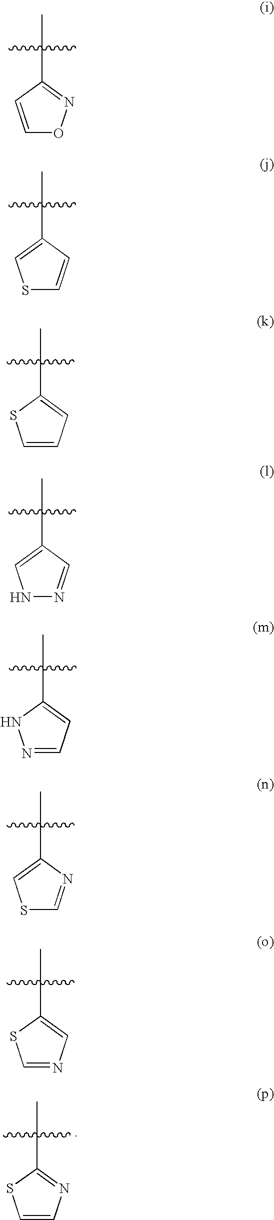

Exemplary embodiments of B include 2-methoxyphenyl, 3-methoxyphenyl, 4-methoxyphenyl, 2,4-dimethoxy-phenyl, 3,4-dimethoxy-phenyl, 3,5-dimethoxy-phenyl, 4-hydroxyphenyl, 3-hydroxyphenyl, 2-hydroxyphenyl, 2-chloro-phenyl, 4-chloro-phenyl, 2,6-dichloro-phenyl, 4-fluoro-phenyl, 3-fluoro-phenyl, 2-fluoro-phenyl, 3,4-difluoro-phenyl, 2,6-difluoro-phenyl, phenyl, 4-butoxy-phenyl, 2-ethoxy-phenyl, 2-nitro-phenyl, 3-nitro-phenyl, 4-nitro-phenyl, 2-trifluoromethoxy-phenyl, 3-trifluoromethoxy-phenyl, 4-trifluoromethoxy-phenyl, 2-trifluoromethyl-phenyl, 4-trifluoromethyl-phenyl, 5-(3-trifluoromethyl-phenyl)-furan-2-yl, 4-benzyloxy-phenyl, 3-methyl-4-trifluoromethyl-phenyl, 2-methyl-phenyl, 3-methyl-phenyl, 4-methyl-phenyl, benzo[1,3]dioxol-5-yl, pyridin-3-yl, pyridin-4-yl, thiophen-2-yl; 2-pyridin-4-yl-phenyl, 2-benzonitrile; 1-phenyl-4-trifluoromethyl-1H-pyrazolyl, 4-bromophenyl; 2-methylsulfanyl-pyridin-3-yl, 2-ethylsulfanyl-pyridin-3-yl, 2-propylsulfanyl-pyridin-3-yl, 2-benzoic acid methyl ester, N-3-phenyl-acetamide, 2-methyl-5-trifluoromethyl-furan-3-yl, 5-Methyl-2-trifluoromethyl-furan-3-yl), 5-tert-butyl-2-methyl-furan-3-yl, 3-chloro-4-fluoro-phenyl, 2,3-dimethyl-phenyl, 2,6-difluoro-3-methyl-phenyl, 2-(4-nitro-phenyl)-5-trifluoromethyl-pyrazolyl-5-yl, 4-tert-butyl-phenyl, 4-dimethylamino-phenyl; cyclohexyl, 4-methoxy-3-trifluoromethyl-phenyl; 2-methyl-3-trifluoromethyl-phenyl, 2-amino-phenyl, 5-(4-methanesulfonyl-phenyl)-furan-2-yl, 2-phenoxy-pyridin-3-yl; 2-difluoromethylsulfanyl-phenyl, N,N-diethyl-4-benzenesulfonamide, 2-phenoxy-phenyl, 2,4,6-trimethyl-phenyl, 2-(4-chloro-phenylsulfanyl)-pyridin-3-yl], 5-chloro-2-trifluoromethyl-phenyl, 5-methyl-2-trifluoromethyl-furan-3-yl, 5-(2,3-dihydro-benzofuran-6-yl)-4-methyl-thiazol-2-yl, 2-fluoro-4-trifluoromethyl-phenyl, 2-fluoro-4-methoxy-phenyl, 2-ethoxy-pyridin-3-yl, 5-methyl-isoxazol-3-yl), 4-benzoic acid, 2,2-difluoro-benzo[1,3]dioxol-5-yl, benzoic acid 2-benzyl ester, 5-benzo[1,3]dioxol-4-yl.

According to one embodiment, R¹ is oxo, 1,2,-methylene dioxy, 1,2-difluoromethylenedioxy, or 1,2-ethylenedioxy.

According to another embodiment, R¹ is R⁶, wherein R⁶ is straight chain or branched (C1-C6)alkyl or (C2-C6 alkenyl) or alkynyl, optionally substituted with R⁷.

According to another embodiment, R¹ is (CH₂)ₙ—Y, wherein n is 0, 1, or 2, and Y is halo, CN, NO₂, CHF₂, CH₂F, CF₃, OCF₃, OCHF₂, SCHF₂, OR⁵, OR⁶, SCHF₂, SR⁵, SR⁶, S(O)R⁶, SO₂R⁶, NH₂, NHR⁶, N(R⁶)₂, NR⁶R⁸, COOH, COOR⁶ or OR⁶. According to one embodiment, Y is halo, CN, NO₂, CF₃, OCF₃, OCHF₂, OR⁵, OR⁶, SCHF₂, SR⁵, SR⁶, S(O)R⁶, SO₂R⁶, NH₂, NHR⁶, N(R⁶)₂, NR⁶R⁸, COOH, COOR⁶ or OR⁶.

According to another embodiment, R¹ is selected from halo, CH₂F, CHF₂, CF₃, NH₂, NH(C1-C4 alkyl), NHC(O)CH₃, OH, O(C1-C4 alkyl), OPh, O-benzyl, SCHF₂, S—(C1-C4 alkyl), C1-C4 alkyl, NO₂, CN, methylenedioxy, ethylenedixoy, SO₂NH(C1-C4 alkyl), or SO₂N(C1-C4 alkyl)₂.

According to another preferred embodiment, R¹ is selected from methyl, n-propyl, i-propyl, t-butyl, halo, CF₃, NH₂, NH(CH₃), NHC(O)CH₃, OH, OCH₃, O-(n) propyl, O-(n)butyl, N(CH₃)₂, OPh, O-benzyl, S-(ethyl), S-(n)propyl, C(O)OCH₃, COOH, NH2, NHCH₃, N(CH₃)₂, S—CH₃, NO₂, CN, methylenedioxy, SO₂NH(n-propyl), or SO₂N(n-propyl)₂.

According to another embodiment, R² is a straight chain or branched (C1-C6)alkyl or (C2-C6) alkenyl or alkynyl, optionally substituted with R¹, R⁴, or R⁵. More preferably, R² is a straight chain or branched (C1-C4)alkyl or (C2-C4) alkenyl or alkynyl, optionally substituted with R¹, R⁴, or R⁵ as defined hereinabove.

According to another embodiment, R³ is an optionally substituted phenyl, napthyl, C5-C10 heteroaryl or C3-C7 heterocyclyl. More preferably, R³ is an optionally substituted phenyl, C5-C6 heteroaryl, or C3-C6 heterocyclyl.

According to another embodiment, R⁴ is selected from OR⁵, OR⁶, SR⁵, SO₂R⁵, SO₂R⁶, SR⁶, C(O)OR⁵, C(O)OR⁶, C(O)N(R⁶)₂, C(O)N(R⁵)₂, C(O)N(R⁵R⁶), NR⁵COR⁵, NR⁵COR⁶, NR⁶COR⁵, or NR⁶COR⁶. Or, R⁴ is selected from OH, C(O)OMe, NHC(O)Me, C(O)NH₂, C(O)NHMe, C(O)NMe₂, SO₂NMe₂, SO₂NEt₂, NH₂, or NMe₂.

According to another embodiment, R⁵ is C5-C6 cycloalkyl, C6 or C10 aryl, C5-C10 heteroaryl or C3-C7 heterocyclyl, optionally substituted with up to 2 R¹. Or, R⁵ is an optionally substituted cyclohexyl, phenyl, C5-C6 heteroaryl, or C3-C6 heterocyclyl. According to another embodiment, R⁵ is pyridyl, tetrazolyl, phenyl, cyclohexyl, pyrazolyl, or furanyl.

According to one embodiment, R⁶ is H.

According to another embodiment, R⁶ is a straight chain or branched (C1-C6)alkyl or (C2-C6 alkenyl) or alkynyl, optionally substituted with R⁷.

According to another embodiment, R⁶ is a straight chain or branched (C1-C6)alkyl or (C2-C6 alkenyl) or alkynyl.

According to one embodiment, R⁷ is C5-C6 cycloalkyl, phenyl, naphthyl, C5-C10 heteroaryl or C3-C7 heterocyclyl, optionally substituted with straight chain or branched (C1-C6)alkyl or (C2-C6 alkenyl) or alkynyl. Or, R⁷ is C5-C6 cycloalkyl, phenyl, naphthyl, C5-C10 heteroaryl or C3-C7 heterocyclyl, optionally substituted with 1-2-methylenedioxy, 1,2-ethylenedioxy, or (CH₂)ₙ—Z. Or, R⁷ is an optionally substituted cyclohexyl, phenyl, C5-C6 heteroaryl, or C3-C6 heterocyclyl.

Embodiments of Z include those described hereinabove for R¹. According to one embodiment, Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic), or O-aliphatic.

According to one embodiment, R⁸ is C(O)aliphatic, C(O)aryl, arylsulfonyl or alkylsulfonyl. Or, R⁸ is acyl.

According to another embodiment, the methods of the present invention employ compounds of formula (IA):

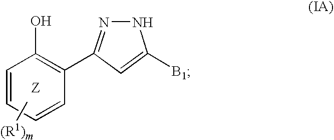

wherein:

m is 0 to 3;

B₁ is selected from:

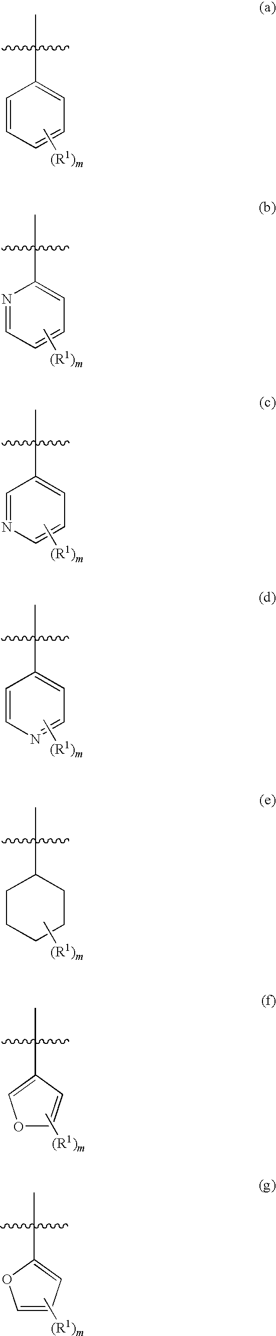

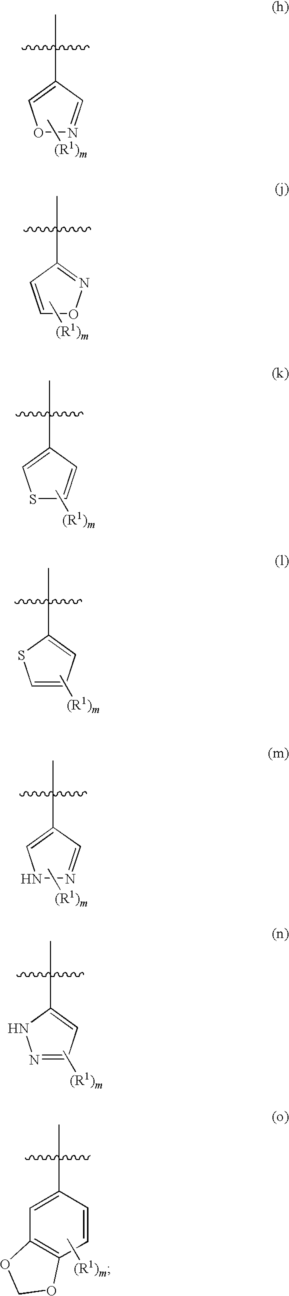

wherein B₁ and ring Z are substituted with up to 2 substituents selected from R², R³, or R⁴; and R¹_{, R}²_{, R}³, or R⁴ are as defined above in formula (I).

According to one embodiment, m is 1 or 2. Or, m is 1. Or, m is 2.

Exemplary embodiments of ring Z, together with the hydroxyl group and optional substituents, include 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl, 2-hydroxy-5-methylsulfonylphenyl, 2-hydroxy-5-amidophenyl, 2-hydroxy-6-methoxyphenyl, 2-hydroxy-4,6-dimethylphenyl, 2-hydroxy-4,5-dimethylphenyl, 2-hydroxy-4-methylphenyl, or 2-hydroxy-4-fluorophenyl.

Exemplary embodiments of B₁ include 2-methoxyphenyl, 3-methoxyphenyl, 4-methoxyphenyl, 2,4-dimethoxy-phenyl, 3,4-dimethoxy-phenyl, 3,5-dimethoxy-phenyl, 4-hydroxyphenyl, 3-hydroxyphenyl, 2-hydroxyphenyl, 2-chloro-phenyl, 4-chloro-phenyl, 2,6-dichloro-phenyl, 4-fluoro-phenyl, 3-fluoro-phenyl, 2-fluoro-phenyl, 3,4-difluoro-phenyl, 2,6-difluoro-phenyl, phenyl, 4-butoxy-phenyl, 2-ethoxy-phenyl, 2-nitro-phenyl, 3-nitro-phenyl, 4-nitro-phenyl, 2-trifluoromethoxy-phenyl, 3-trifluoromethoxy-phenyl, 4-trifluoromethoxy-phenyl, 2-trifluoromethyl-phenyl, 4-trifluoromethyl-phenyl, 5-(3-trifluoromethyl-phenyl)-furan-2-yl, 4-benzyloxy-phenyl, 3-methyl-4-trifluoromethyl-phenyl, 2-methyl-phenyl, 3-methyl-phenyl, 4-methyl-phenyl, benzo[1,3]dioxol-5-yl, pyridin-3-yl, pyridin-4-yl, thiophen-2-yl, 2-pyridin-4-yl-phenyl, 2-benzonitrile; 1-phenyl-4-trifluoromethyl-1H-pyrazolyl, 4-bromophenyl; 2-methylsulfanyl-pyridin-3-yl, 2-ethylsulfanyl-pyridin-3-yl, 2-propylsulfanyl-pyridin-3-yl, 2-benzoic acid methyl ester, N-3-phenyl-acetamide, 2-methyl-5-trifluoromethyl-furan-3-yl, 5-Methyl-2-trifluoromethyl-furan-3-yl), 5-tert-butyl-2-methyl-furan-3-yl, 3-chloro-4-fluoro-phenyl, 2,3-dimethyl-phenyl, 2,6-difluoro-3-methyl-phenyl, 2-(4-nitro-phenyl)-5-trifluoromethyl-pyrazolyl-5-yl, 4-tert-butyl-phenyl, 4-dimethylamino-phenyl; cyclohexyl, 4-methoxy-3-trifluoromethyl-phenyl; 2-methyl-3-trifluoromethyl-phenyl, 2-amino-phenyl, 5-(4-methanesulfonyl-phenyl)-furan-2-yl, 2-phenoxy-pyridin-3-yl; 2-difluoromethylsulfanyl-phenyl, N,N-diethyl-4-benzenesulfonamide, 2-phenoxy-phenyl, 2,4,6-trimethyl-phenyl, 2-(4-chloro-phenylsulfanyl)-pyridin-3-yl], 5-chloro-2-trifluoromethyl-phenyl, 5-methyl-2-trifluoromethyl-furan-3-yl, 5-(2,3-dihydro-benzofuran-6-yl)-4-methyl-thiazol-2-yl, 2-fluoro-4-trifluoromethyl-phenyl, 2-fluoro-4-methoxy-phenyl, 2-ethoxy-pyridin-3-yl, 5-methyl-isoxazol-3-yl), 4-benzoic acid, 2,2-difluoro-benzo[1,3]dioxol-5-yl, benzoic acid 2-benzyl ester, 5-benzo[1,3]dioxol-4-yl.

According to another embodiment, the methods of the present invention are practiced using a compound of formula (IA′):

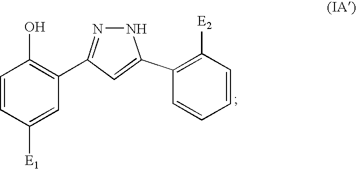

wherein m is 0-3; and

each of E₁ and E₂ is independently an electronegative group.

The term “electronegative group” as used herein has a meaning well known in the art. See, e.g., March, Advanced Organic Chemistry, 4^{th} Ed., John Wiley & Sons, 1992, the disclosure whereof is incorporated herein by reference. Embodiments of E₁ and E₂ include those groups within R¹, R², R³, R⁴, and R⁵ that are electronegative. Examples of such groups are halo, CF₃, CONH₂, SO₂NEt₂, CN, COOH, COO-(aliphatic), SO₂(aliphatic), SO₂(aryl), etc.

According to another embodiment, the present invention provides a compound having formula (II):

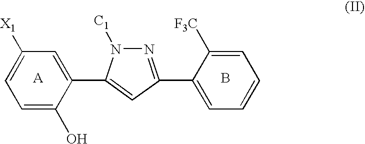

or a pharmaceutically acceptable salt thereof, wherein:

C₁ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

X₁ is selected from halo, R², CF₃, CN, COOH, COOR, C(O)R, C(O)NH₂, C(O)NHR, or C(O)N(R)₂;

each R is independently R² or R³;

wherein each of ring B, optionally including X₁ and OH, and C₁ optionally comprises up to 4 substituents, and ring A optionally comprises up to 3 substituents, wherein said substituents are independently selected from R¹, R²_{, R}³_{, R}⁴, or R⁵;

R¹ is R⁶ or (CH₂)ₙ—Y;

n is 0, 1 or 2;

Y is halo, CN, NO₂, CF₃, CHF₂, CH₂F, OCF₃, OH, SCHF₂, SR⁶, S(O)R⁶, SO₂R⁶, NH₂, NHR⁶, N(R⁶)₂, NR⁶R⁸, COOH, COOR⁶ or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R²_{, R}⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N (R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CHF₂, CH₂F, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic), or O-aliphatic; and

R⁸ is an amino protecting group.

Embodiments of R¹, R², R³, R⁴, R⁵, and R⁶ in formula (II) include those described hereinabove for compounds of formula (I). Compounds of formula (II) are, e.g., useful in the methods of the present invention.

Embodiments of C₁ include those described above for radical C in formula (I) above. According to one embodiment, C₁ is H.

According to another embodiment, X₁ is selected from (C1-C4)-aliphatic, or C(O)—NH₂.

Compounds of formula (II) include those having one or more, or, more preferably, all, of the features selected from the group:

(a) X₁ is chloro, fluoro, CF₃, CN, COOH, CONH₂, CONHR₂; and

(b) C₁ is H or phenyl.

Compounds of formula (II) include IA-6 in Table 1.

According to another embodiment, the present invention provides a compound having formula (III):

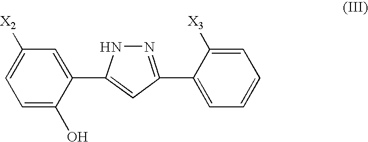

or a pharmaceutically acceptable salt thereof, wherein:

X₂ is selected from halo, R², CF₃, CN, COOH, COOR², COOR³, C(O)R², C(O)R³, C(O)NH₂, C(O)NHR, or C(O)NR²;

X₃ is selected from H, halo, CF₃, or NO₂;

each R is independently R² or R³;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶ or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R²_{, R}⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N (R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CHF₂, CH₂F, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group; provided that:

(i) when X₃ is H, then X₂ is not methyl, chloro, or bromo;

(ii) when X₂ is chloro, then X₃ is not fluoro, chloro, or nitro;

(iii) when X₂ is methyl, then X₃ is not nitro or chloro.

Embodiments of R¹, R², R³, R⁴, R⁵, and R⁶ in formula (III) include those described hereinabove for compounds of formula (I). Compounds of formula (III) are, e.g., useful in the methods of the present invention.

Compounds of formula (III) include those having one or more, or, more preferably, all, of the features selected from the group:

(a) X₃ is halo, CF₃, or NO₂; and

(b) X₂ is halo, CF₃, methyl, ethyl, propyl, or CONH₂.

Exemplary compounds of formula (III) include IA-6, IA-20, IA-26 of Table 1.

According to another embodiment, the present invention provides a compound having formula (IV):

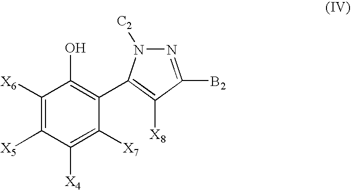

or a pharmaceutically acceptable salt thereof;
wherein:

B₂ is selected from:

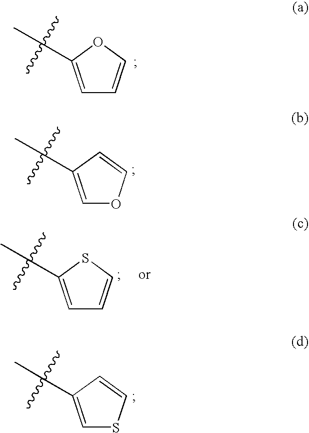

C₂ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

each of X₄, X₅, X₆, X₇, and X₈ is selected from (CH₂)ₙ—Y, R², R³, R⁴, R⁵ or R⁶;

wherein each of B₂ and C₂ optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R²_{, R}⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O) (OR⁶)₂, OP(O)(OR⁵)₂, OP(O)(OR⁶)(OR⁵), SR⁶, SR⁵. S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O) (OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic), or O-aliphatic; and

R⁸ is an amino protecting group; provided that:
(i) when B₂ is structure (a), X₅, X₆, and C₂ are H, then X₄ is not H, Cl, CH₃, or OCH₃;
(ii) when B₂ is structure (c), X₅, X₆, and C₂ is H, then X₄ is not H or CH₃;
(iii) when B₂ is structure (a), X₄ is Cl or CH₃, X₅ and C₂ are H, then X₆ is not NO₂, Cl, or Br;
(iv) when B₂ is structure (a), X₄ is Cl, X₅ and X₆ are H, then C₂ is not Ph, —C(O)CH3, —C(O)Ph, or —C(O)NHPh;
(v) when B₂ is structure (a), X₄ is CH₃, X₅ and X₆ is H; then C₂ is not Ph;
(vi) when B₂ is structure (a), X₄, X₅, and X₆ is H, then C₂ is not CH₃, C(O)CH₃, or —C(O)—NHPh;
(vii) when B₂ is structure (c), X₄, X₅, and X₆ is H, then C₂ is not CH₃ or C(O)CH₃;
(viii) when B₂ is structure (a), X₄ is Cl, X₅ is H, X₆ is NO₂ or Br, then X₂ is not Ph, C(O)CH₃, or C(O)Ph.

Embodiments of R¹, R², R³, R⁴, R⁵, and R⁶ in formula (IV) include those described hereinabove for compounds of formula (I). Compounds of formula (IV) are, e.g., useful in the methods of the present invention.

According to one embodiment, B₂ is optionally substituted ring

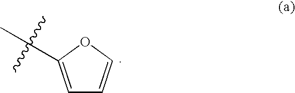

According to one embodiment, B₂ is optionally substituted ring

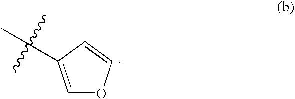

According to one embodiment, B₂ is optionally substituted ring

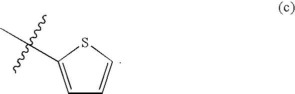

According to one embodiment, B₂ is optionally substituted ring

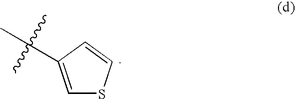

Embodiments of C₂ include those described above for radical C in formula (I). According to another embodiment, C₂ is H or phenyl. Or, C₂ is H.

Embodiments of X₈ include those described hereinabove for radical X in formula (I). According to another embodiment, X₈ is H or phenyl. Or, X₈ is H.

Compounds of formula (IV) include those having one or more, or, more preferably, all, of the features selected from the group:
(a) B₂ is:
5-(3′-trifluoromethylphenyl)-furan-2-yl;
5-trifluoromethyl-2-methyl-furan-3-yl;
5-t-butyl-2-methyl-furan-3-yl;
5-methyl-2-trifluoromethyl-furan-3-yl; or
5-(4′-methylsulfonylphenyl)-furan-2-yl;
   (b) C₂ is H or phenyl;
   (c) X₄ is halo, (C1-C4)alkyl, CF₃, CN, or NO₂;
   (d) X₅, X₆, and X₇ are H; and
   (e) X₈ is H.

According to another embodiment, X₄, X₅, X₆, and X₇, taken together with the hydroxyphenyl group, is selected from 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl 2-hydroxy-5-methylsulfonylphenyl, or 2-hydroxy-5-amidophenyl.

Compounds of formula (IV) include IA-7, IA-28, IA-42, IA-50, IA-64, IA-76, and IA-92 of Table 1.

According to another embodiment, the present invention provides a compound of formula (V):

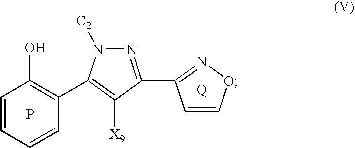

or a pharmaceutically acceptable salt thereof;

wherein:

C₃ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

X₉ is selected from (CH₂)ₙ—Y, R²_{, R}³, R⁴, R⁵ or R⁶;

wherein each of ring P, optionally including the hydroxyl group, and ring Q optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R², R⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CHF₂, CH₂F, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group.

Embodiments of R¹, R², R³, R⁴, R⁵, and R⁶ included in formula (V) are those described hereinabove for compounds of formula (I). Compounds of formula (V) are, e.g., useful in the methods of the present invention.

Embodiments of C₃ include those described hereinabove for radical C in formula (I). According to one embodiment, C₃ is H or phenyl. Or, C₃ is H.

Embodiments of X₉ include those described hereinabove for radical X in formula (I). According to another embodiment X₉ is H or phenyl. Or, X₉ is H.

According to another embodiment, ring P, together with the 2-hydroxy group is a 2-hydroxy-5-substituted phenyl ring.

Compounds of formula (V) include those having one or more, or, more preferably, all, of the features selected from the group:
(a) C₃ is H or phenyl;
(b) ring Q is isoxazol-3-yl or 5-methyl-isoxazol-3-yl;
(c) X₉ is H; and
(d) ring P together with the hydroxy substituent is selected from:
2-hydroxy-5-methoxyphenyl,
2-hydroxy-5-methylphenyl,
2-hydroxy-5-fluorophenyl,
2-hydroxy-5-ethylphenyl,
2-hydroxy-5-propylphenyl,
2-hydroxy-5-chlorophenyl,
2-hydroxy-5-isopropylphenyl,
2-hydroxy-5-tetrazol-2H-3-ylphenyl,
2-hydroxy-5-bromophenyl,
2-hydroxy-5-methylsulfonylphenyl, or
2-hydroxy-5-amidophenyl.

Compounds of formula (V) include IA-107 of Table 1.

According to another embodiment, the present invention provides a compound having formula (VI):

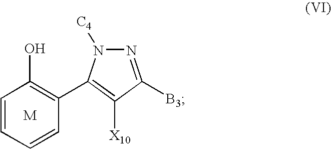

or a pharmaceutically acceptable salt thereof;

wherein:
B₃ is selected from:

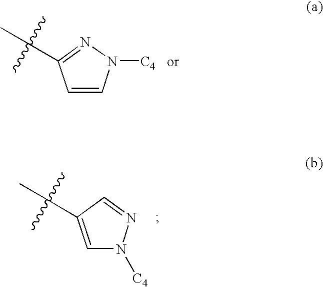

C₄ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

X₁₀ is selected from (CH₂)ₙ—Y, R², R³, R⁴, R⁵ or R⁶;

wherein each of ring M, optionally including the hydroxyl group, C₄, and B₃ optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R²_{, R}⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group.

Embodiments of R¹, R², R³, R⁴, R⁵, and R⁶ in formula (VI) include those described hereinabove for compounds of formula (I). Compounds of formula (VI) are, e.g., useful in the methods of the present invention.

Embodiments of C₄ include those described hereinabove for radical C in formula (I). According to one embodiment, C₄ is H or phenyl. Or, C₄ is H.

According to another embodiment, X₁₀ is H or phenyl. Or, X₁₀ is H.

According to one embodiment, B₃ is optionally substituted

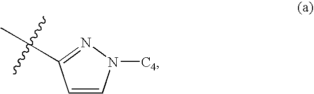

wherein C₄ is as defined above.

According to another embodiment, B₃ is optionally substituted

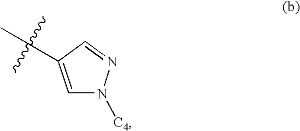

wherein C₄ is as defined above.

According to another embodiment ring M, together with the 2-hydroxy group, is a 2-hydroxy-5-substituted phenyl ring. Specific embodiments thereof include 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl 2-hydroxy-5-methylsulfonylphenyl, or 2-hydroxy-5-amidophenyl.

Compounds of formula (VI) include IA-31 in Table 1.

According to another embodiment, the present invention provides compounds of formula (VII):

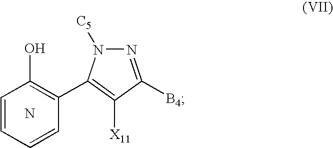

or a pharmaceutically acceptable salt thereof;
wherein:

B₄ is selected from:

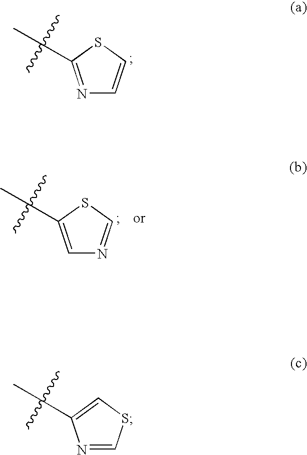

C₅ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

X₁₁ is selected from (CH₂)ₙ—Y, R², R³, R⁴, R⁵ or R⁶;

wherein each of ring N, optionally including the hydroxyl group, C₅, and B₄ optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R², R⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group;

provided that:

(a) when C₅ is H, X₁₁ is H, ring N is 2-hydroxy-4-methoxyphenyl, then B₄ is not 2-methylthiazol-4-yl;

(b) when C₅ is H, X₁₁ is H, ring N is 2-hydroxy-4,5-dimethylphenyl, then B₄ is not 2-methylthiazol-4-yl.

Embodiments of R¹, R², R³, R⁴, R⁵, and R⁶ in formula (VII) include those described hereinabove for compounds of formula (I). Compounds of formula (VII) are, e.g., useful in the methods of the present invention.

Embodiments of C₅ include those described hereinabove for radical C in formula (I). According to one embodiment, C₅ is H or phenyl. Or, C₅ is H.

Embodiments of X₁₁ include those described hereinabove for radical X in formula (I). According to another embodiment, X₁₁ is H or phenyl. Or, X₁₁ is H.

According to another embodiment, B₄ is optionally substituted

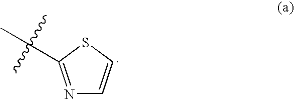

According to another embodiment, B₄ is optionally substituted

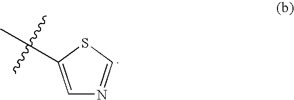

According to another embodiment, B₄ is optionally substituted

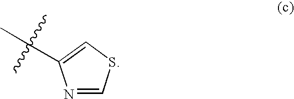

According to another embodiment, ring N, together with the 2-hydroxy group, is a 2-hydroxy, 5-substituted phenyl ring. Exemplary compounds of ring N, together with the 2-hydroxy group, include 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl, 2-hydroxy-5-methylsulfonylphenyl, 2-hydroxy-5-amidophenyl, 2-hydroxy-6-methoxyphenyl, 2-hydroxy-4,6-dimethylphenyl, 2-hydroxy-4,5-dimethylphenyl, 2-hydroxy-4-methylphenyl, or 2-hydroxy-4-fluorophenyl.

Compounds of formula (VII) include IA-95 in Table 1.

According to another embodiment, the present invention provides a compound of formula (VIII):

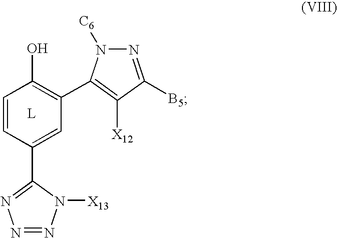

or a pharmaceutically acceptable salt thereof, wherein:
B₅ is optionally substituted aryl, heteroaryl, cycloaliphatic, or heterocyclyl;

C₆ and X₁₃ each is independently selected from H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

X₁₂ is selected from (CH₂)ₙ—Y, R², R³, R⁴, R⁵ or R⁶;

wherein each of ring L, including the hydroxyl group, C₆, and B₅ optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R², R⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group.

Embodiments of R¹, R², R³, R⁴, R⁵, and R⁶ in formula (VIII) include those described hereinabove for compounds of formula (I). Compounds of formula (VIII) are, e.g., useful in the methods of the present invention.

Embodiments of C₆ include those described hereinabove for radical C in formula (I). According to one embodiment, C₆ is H or phenyl. Or, C₆ is phenyl.

Embodiments of X₁₂ include those described hereinabove for radical X in formula (I). According to another embodiment, each of X₁₂ and X₁₃ is H or phenyl. Or, each is independently H.

According to another embodiment, B₅ is optionally substituted aryl. Or, B₅ is optionally substituted phenyl. Or, B₅ is phenyl.

According to another embodiment, B₅ is optionally substituted heteroaryl. Or, B₅ is optionally substituted pyridyl, furanyl, thiophenyl, thiazolyl, isoxazolyl, or pyrazolyl.

According to another embodiment, B₅ is cycloaliphatic. Or, B₅ is cyclohexyl or cyclopentyl. Or, B₅ is heterocyclyl.

Exemplary embodiments of B₅ include 2-methoxyphenyl, 3-methoxyphenyl, 4-methoxyphenyl, 2,4-dimethoxy-phenyl, 3,4-dimethoxy-phenyl, 3,5-dimethoxy-phenyl, 4-hydroxyphenyl, 3-hydroxyphenyl, 2-hydroxyphenyl, 2-chloro-phenyl, 4-chloro-phenyl, 2,6-dichloro-phenyl, 4-fluoro-phenyl, 3-fluoro-phenyl, 2-fluoro-phenyl, 3,4-difluoro-phenyl, 2,6-difluoro-phenyl, phenyl, 4-butoxy-phenyl, 2-ethoxy-phenyl, 2-nitro-phenyl, 3-nitro-phenyl, 4-nitro-phenyl, 2-trifluoromethoxy-phenyl, 3-trifluoromethoxy-phenyl, 4-trifluoromethoxy-phenyl, 2-trifluoromethyl-phenyl, 4-trifluoromethyl-phenyl, 5-(3-trifluoromethyl-phenyl)-furan-2-yl, 4-benzyloxy-phenyl, 3-methyl-4-trifluoromethyl-phenyl, 2-methyl-phenyl, 3-methyl-phenyl, 4-methyl-phenyl, benzo[1,3]dioxol-5-yl, pyridin-3-yl, pyridin-4-yl, thiophen-2-yl, 2-pyridin-4-yl-phenyl, 2-benzonitrile; 1-phenyl-4-trifluoromethyl-1H-pyrazolyl, 4-bromophenyl; 2-methylsulfanyl-pyridin-3-yl, 2-ethylsulfanyl-pyridin-3-yl, 2-propylsulfanyl-pyridin-3-yl, 2-benzoic acid methyl ester, N-3-phenyl-acetamide, 2-methyl-5-trifluoromethyl-furan-3-yl, 5-Methyl-2-trifluoromethyl-furan-3-yl), 5-tert-butyl-2-methyl-furan-3-yl, 3-chloro-4-fluoro-phenyl, 2,3-dimethyl-phenyl, 2,6-difluoro-3-methyl-phenyl, 2-(4-nitro-phenyl)-5-trifluoromethyl-pyrazolyl-5-yl, 4-tert-butyl-phenyl, 4-dimethylamino-phenyl; cyclohexyl, 4-methoxy-3-trifluoromethyl-phenyl; 2-methyl-3-trifluoromethyl-phenyl, 2-amino-phenyl, 5-(4-methanesulfonyl-phenyl)-furan-2-yl, 2-phenoxy-pyridin-3-yl; 2-difluoromethylsulfanyl-phenyl, N,N-diethyl-4-benzenesulfonamide, 2-phenoxy-phenyl, 2,4,6-trimethyl-phenyl, 2-(4-chloro-phenylsulfanyl)-pyridin-3-yl], 5-chloro-2-trifluoromethyl-phenyl, 5-methyl-2-trifluoromethyl-furan-3-yl, 5-(2,3-dihydro-benzofuran-6-yl)-4-methyl-thiazol-2-yl, 2-fluoro-4-trifluoromethyl-phenyl, 2-fluoro-4-methoxy-phenyl, 2-ethoxy-pyridin-3-yl, 5-methyl-isoxazol-3-yl), 4-benzoic acid, 2,2-difluoro-benzo[1,3]dioxol-5-yl, benzoic acid 2-benzyl ester, 5-benzo[1,3]dioxol-4-yl.

Compounds of formula (VIII) include IA-54 in Table 1.

According to another embodiment, the present invention provides a compound of formula (IX):

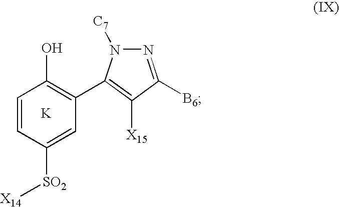

or a pharmaceutically acceptable salt thereof, wherein:

B₆ is phenyl;

C₇ is selected from H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

X₁₄ is R², R³, NHR², NHR³, NR², R³, N(R²)₂;

X₁₅ is selected from (CH₂)ₙ—Y, R², R³, R⁴, R⁵ or R⁶;

wherein each of ring K, optionally including the hydroxyl group, C₇, and B₆ optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R², R⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂(R⁶)₂, SO₂(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵) R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group.

Embodiments of R¹, R², R³, R⁴, R⁵, and R⁶ in formula (IX) include those described hereinabove for compounds of formula (I). Compounds of formula (IX) are, e.g., useful in the methods of the present invention.

Embodiments of C₇ include those described hereinabove for radical C in formula (I). According to one embodiment, C₇ is H or phenyl. Or, C₇ is phenyl.

According to another embodiment, X₁₅ is H or phenyl. Or, X₁₅ is phenyl.

According to another embodiment, X₁₄ is selected from optionally substituted (C1-C6)aliphatic, aryl, NH(C1-C6)aliphatic, NH(aryl), or NH₂. Preferred X₁₄ include optionally substituted (C1-C4)-alkyl, phenyl, NH[(C1-C4)-alkyl], NH(phenyl), or NH₂.

According to one embodiment, B₆ is optionally substituted with up to 2 substituents. Exemplary embodiments of B₆ include 2-methoxyphenyl, 3-methoxyphenyl, 4-methoxyphenyl, 2,4-dimethoxy-phenyl, 3,4-dimethoxy-phenyl, 3,5-dimethoxy-phenyl, 4-hydroxyphenyl, 3-hydroxyphenyl, 2-hydroxyphenyl, 2-chloro-phenyl, 4-chloro-phenyl, 2,6-dichloro-phenyl, 4-fluoro-phenyl, 3-fluoro-phenyl, 2-fluoro-phenyl, 3,4-difluoro-phenyl, 2,6-difluoro-phenyl, phenyl, 4-butoxy-phenyl, 2-ethoxy-phenyl, 2-nitro-phenyl, 3-nitro-phenyl, 4-nitro-phenyl, 2-trifluoromethoxy-phenyl, 3-trifluoromethoxy-phenyl, 4-trifluoromethoxy-phenyl, 2-trifluoromethyl-phenyl, 4-trifluoromethyl-phenyl, 5-(3-trifluoromethyl-phenyl)-furan-2-yl, 4-benzyloxy-phenyl, 3-methyl-4-trifluoromethyl-phenyl, 2-methyl-phenyl, 3-methyl-phenyl, 4-methyl-phenyl, benzo[1,3]dioxol-5-yl, pyridin-3-yl, pyridin-4-yl, 2-benzonitrile, 1-phenyl-4-trifluoromethyl-1H-pyrazolyl, 4-bromophenyl, 2-benzoic acid methyl ester, N-3-phenyl-acetamide, 3-chloro-4-fluoro-phenyl, 2,3-dimethyl-phenyl, 2,6-difluoro-3-methyl-phenyl, 4-tert-butyl-phenyl, 4-dimethylamino-phenyl, 4-methoxy-3-trifluoromethyl-phenyl, 2-methyl-3-trifluoromethyl-phenyl, 2-amino-phenyl, 5-(4-methanesulfonyl-phenyl)-furan-2-yl, 2-difluoromethyl sulfanyl-phenyl, N,N-diethyl-4-benzenesulfonamide, 2-phenoxy-phenyl, 2,4,6-trimethyl-phenyl, 5-chloro-2-trifluoromethyl-phenyl, 2-fluoro-4-trifluoromethyl-phenyl, 2-fluoro-4-methoxy-phenyl, 4-benzoic acid, 2,2-difluoro-benzo[1,3]dioxol-5-yl, benzoic acid 2-benzyl ester.

Compounds of formula (IX) include IA-61 in Table 1.

According to another embodiment, the present invention provides a compound of formula (X):

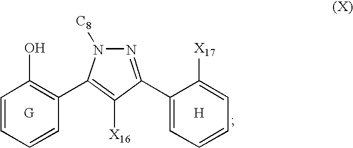

or a pharmaceutically acceptable salt thereof;
wherein:

C₈ is selected from H, aryl, heterocyclic, heteroaryl, aliphatic, C(O)R², C(O)R³, C(O)NH₂, C(O)NHR², C(O)NHR³, C(O)N(R²)₂, C(O)N(R³)₂;

X₁₆ is selected from selected from (CH₂)ₙ—Y, R², R³, R⁴, R⁵ or R⁶;

X₁₇ is CN, tetrazolyl, SO₂R², SO₂R³, SO₂NHR², SO₂NHR³, SO₂NR²R³, SO₂N(R²)₂;

wherein each of ring G, optionally including the hydroxyl group, C₈, and ring H optionally comprises up to 4 substituents independently selected from R¹, R², R³, R⁴, or R⁵;

R¹ is oxo, R⁶ or (CH₂)ₙ—Y;

n is 0, 1 or 2;

Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or

two R¹ on adjacent ring atoms, taken together, form 1,2-methylenedioxy, 1,2-difluoromethylenedioxy, or 1,2-ethylenedioxy;

R² is aliphatic, wherein each R² optionally comprises up to 2 substituents independently selected from R¹, R⁴, or R⁵;

R³ is a cycloaliphatic, aryl, heterocyclic, or heteroaryl ring optionally comprising up to 3 substituents, independently selected from R¹, R², R⁴ or R⁵;

R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);

R⁵ is a cycloaliphatic, aryl, heterocyclic, or heteroaryl ring optionally comprising up to 3 R¹ substituents;

R⁶ is H or aliphatic, wherein R⁶ optionally comprises a R⁷ substituent;

R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;

Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and

R⁸ is an amino protecting group.

Embodiments of R¹, R², R³, R⁴, R⁵, and R⁶ in formula (X) include those described hereinabove for compounds of formula (I). Compounds of formula (X) are, e.g., useful in the methods of the present invention.

Embodiments of C₈ include those described hereinabove for radical X in formula (I). According to one embodiment, C₈ is H or phenyl. Or, C₈ is H.

According to another embodiment, X₁₆ is H or phenyl. Or, X₁₆ is H.

According to another embodiment, X₁₇ is CN, SO₂[(C1-C6)aliphatic], SO₂(aryl), SO₂NH[(C1-C6)aliphatic], SO₂NH(aryl). An exemplary aryl group is optionally substituted phenyl.

According to another embodiment, ring G together with the 2-hydroxy group is 2-hydroxy-5-substituted phenyl.

Exemplary compounds of formula (I) are shown below in Table 1 and Table 2:

TABLE 1 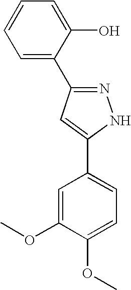
IA-1 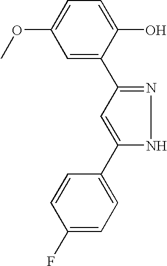
IA-2 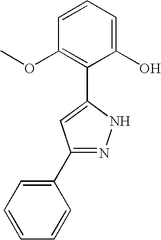
IA-3 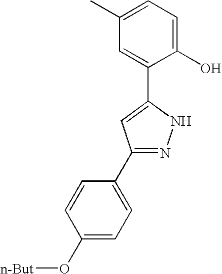
IA-4 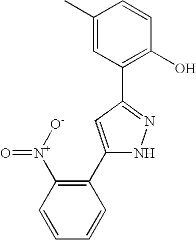
IA-5 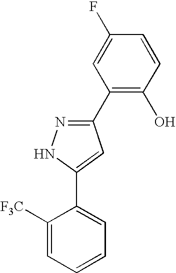
IA-6 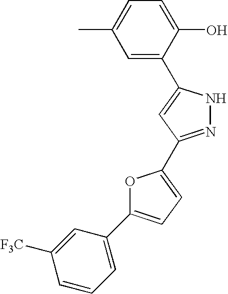
IA-7 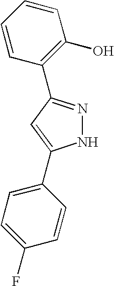
IA-8 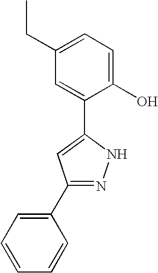
IA-9 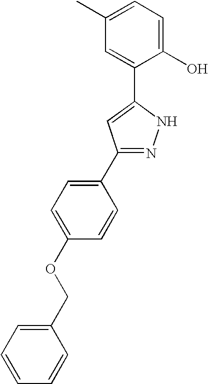
IA-10 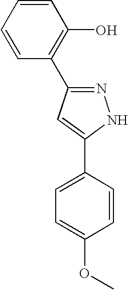
IA-11 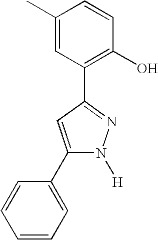
IA-12 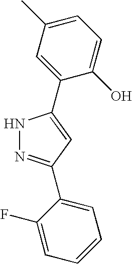
IA-13 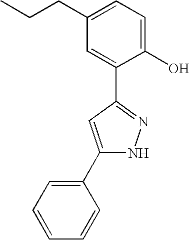
IA-14 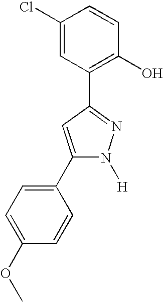
IA-15 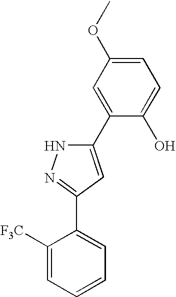
IA-16 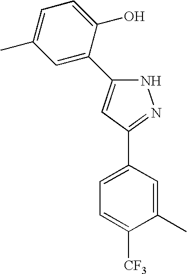
IA-17 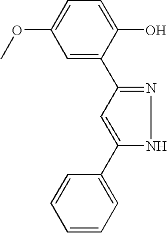
IA-18 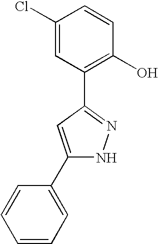
IA-19 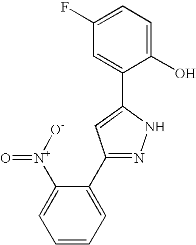
IA-20 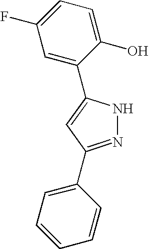
IA-21 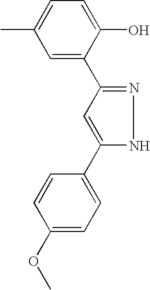
IA-22 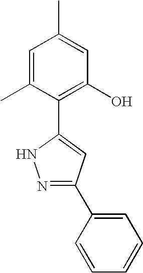
IA-23 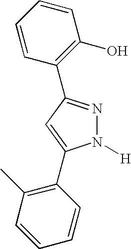
IA-24 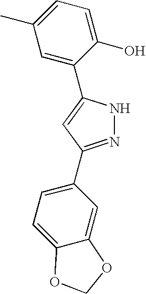
IA-25 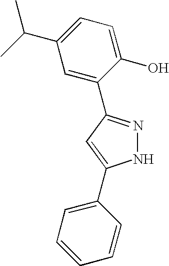
IA-26 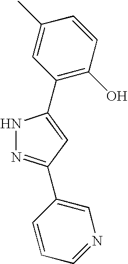
IA-27 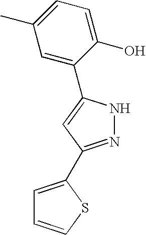
IA-28 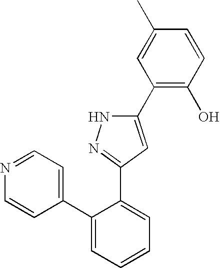
IA-29 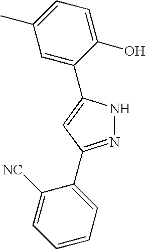
IA-30 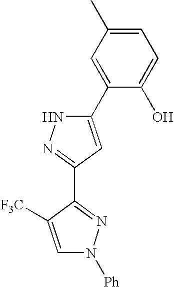
IA-31 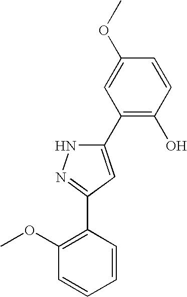
IA-32 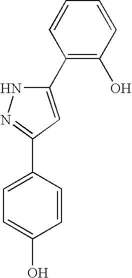
IA-33 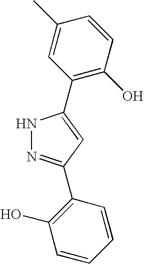
IA-34 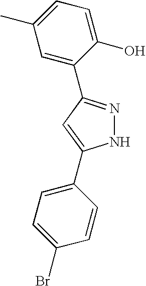
IA-35 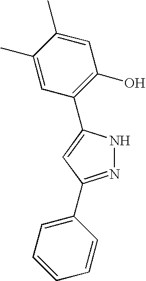
IA-36 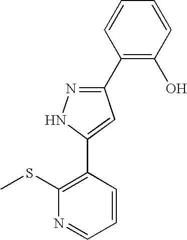
IA-37 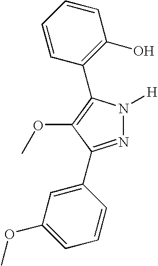
IA-38 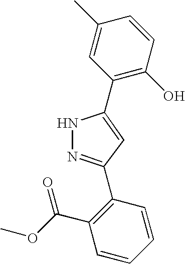
IA-39 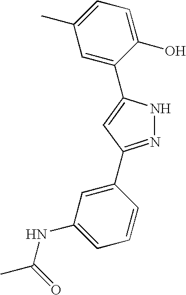
IA-40 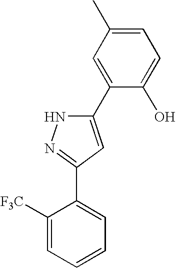
IA-41 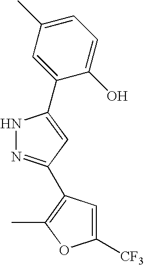
IA-42 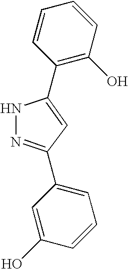
IA-43 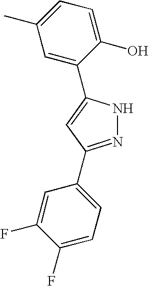
IA-44 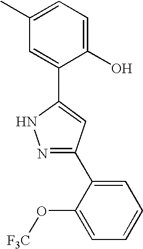
IA-45 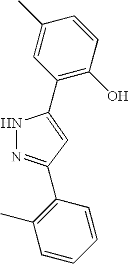
IA-46 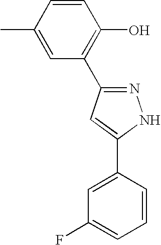
IA-47 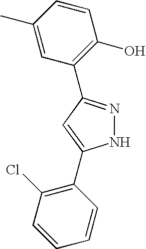
IA-48 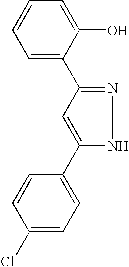
IA-49 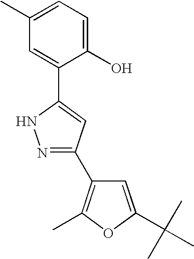
IA-50 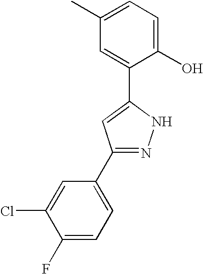
IA-51 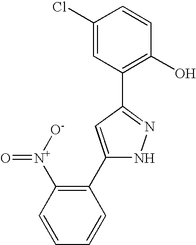
IA-52 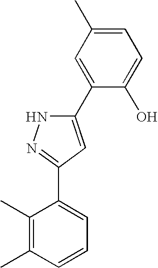
IA-53 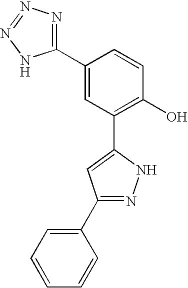
IA-54 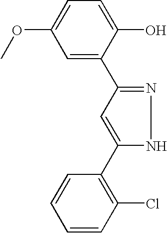
IA-55 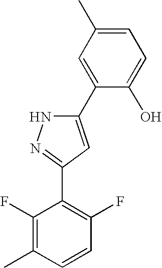
IA-56 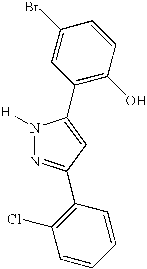
IA-57 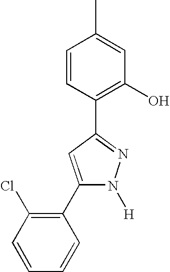
IA-58 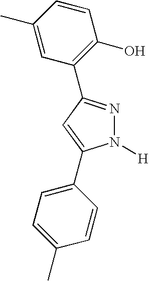
IA-59 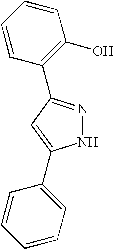
IA-60 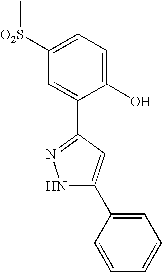
IA-61 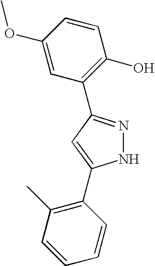
IA-62 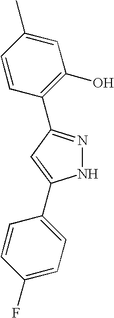
IA-63 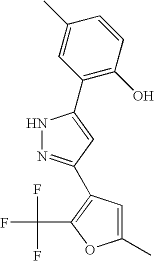
IA-64 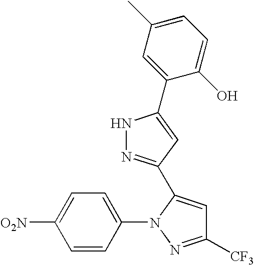
IA-65 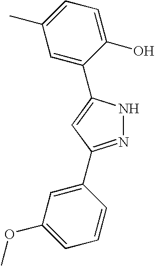
IA-66 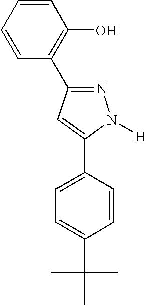
IA-67 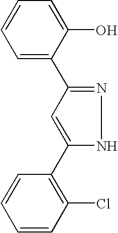
IA-68 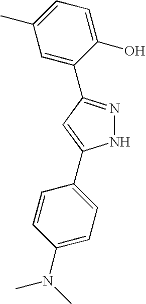
IA-69 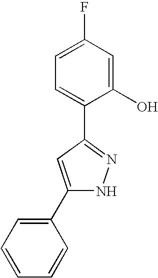
IA-70 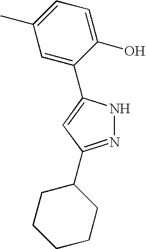
IA-71 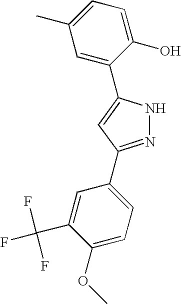
IA-72 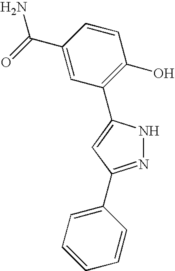
IA-73 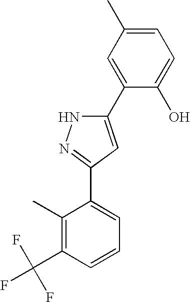
IA-74 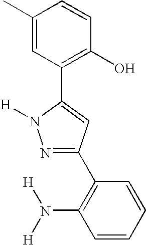
IA-75 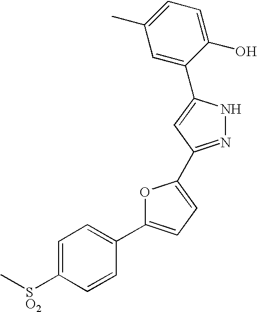
IA-76 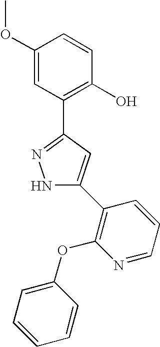
IA-77 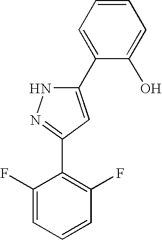
IA-78 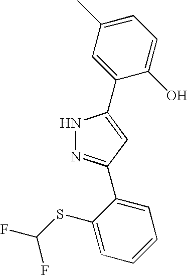
IA-79 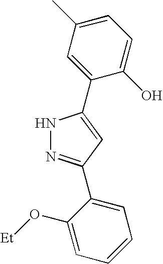
IA-80 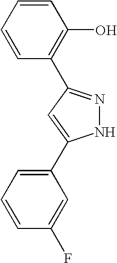
IA-81 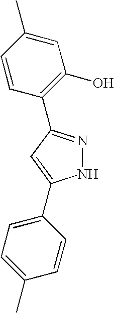
IA-82 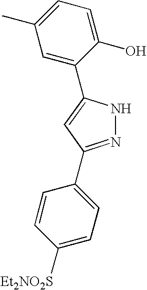
IA-83 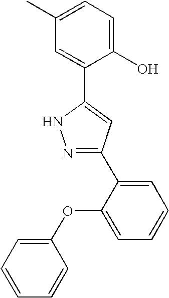
IA-84 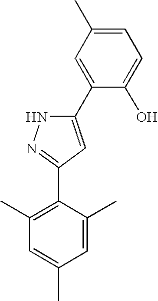
IA-85 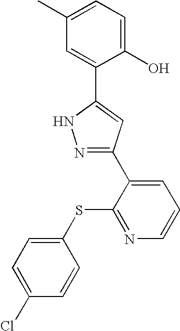
IA-86 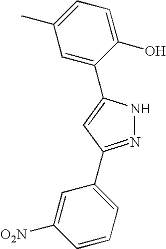
IA-87 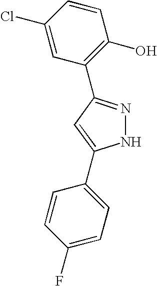
IA-88 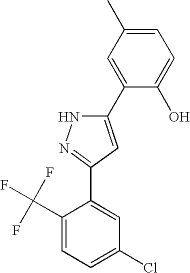
IA-89 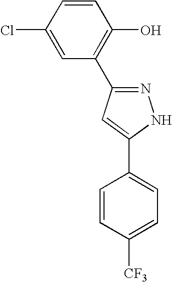
IA-90 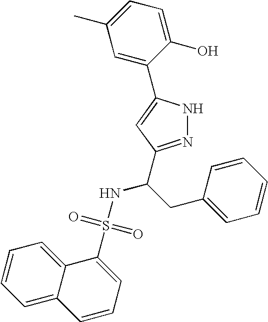
IA-91 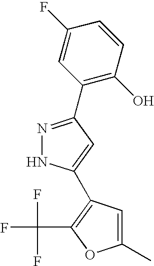
IA-92 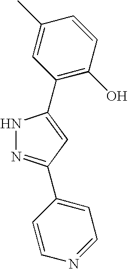
IA-93 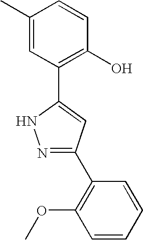
IA-94 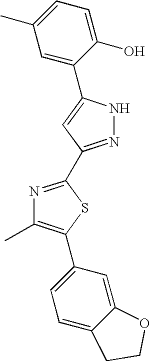
IA-95 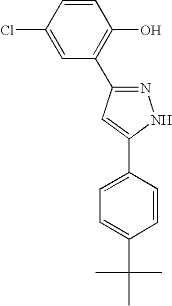
IA-96 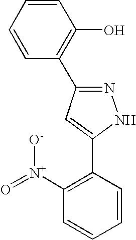
IA-97 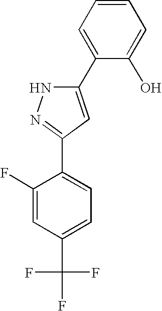
IA-98 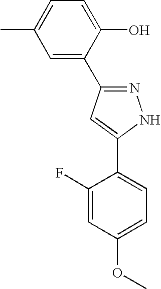
IA-99 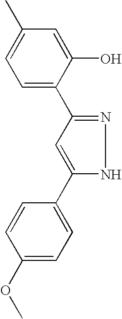
IA-100 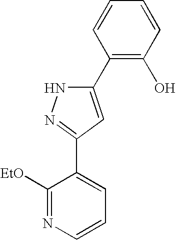
IA-101 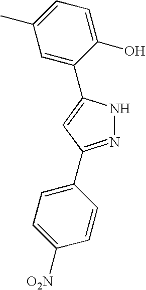
IA-102 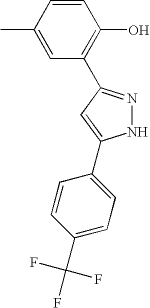
IA-103 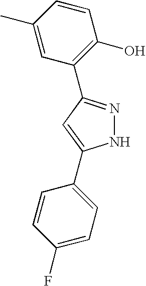
IA-104 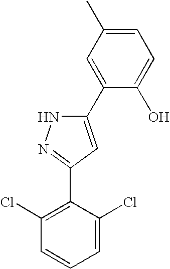
IA-105 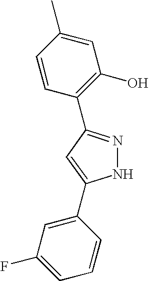
IA-106 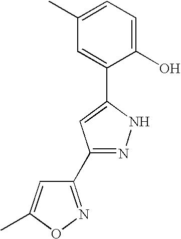
IA-107 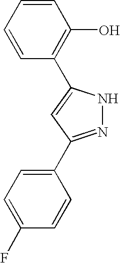
IA-108 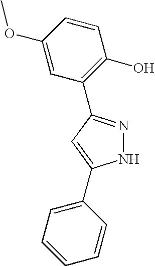
IA-109 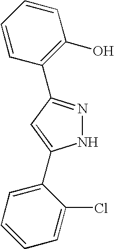
IA-110 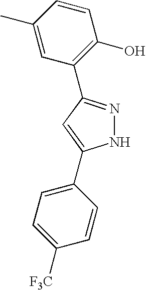
IA-111 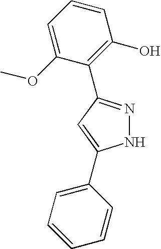
IA-112 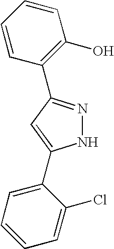
IA-113 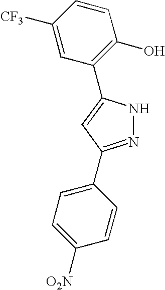
IA-114 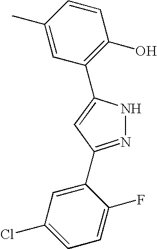
IA-115 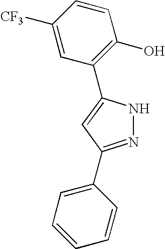
IA-116 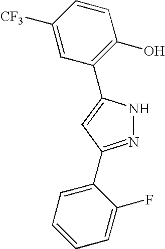
IA-117 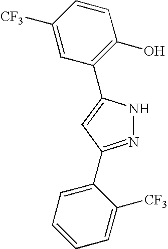
IA-118 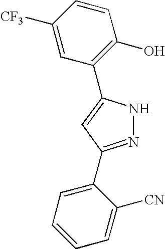
IA-119 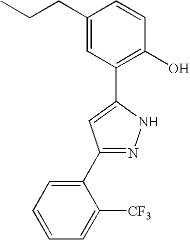
IA-120 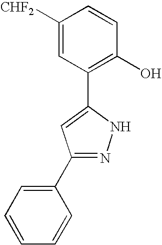
IA-121 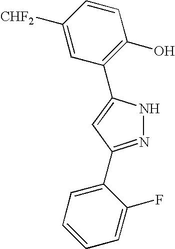
IA-122 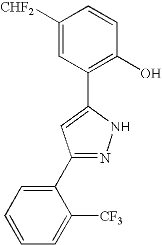
IA-123 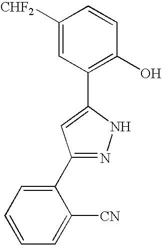
IA-124 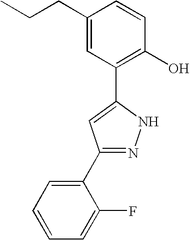
IA-125 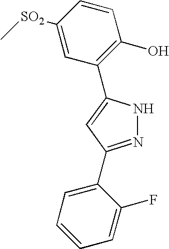
IA-126 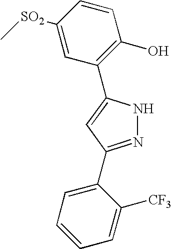
IA-127 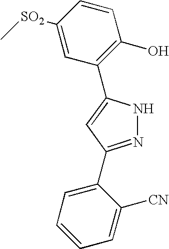
IA-128 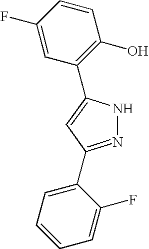
IA-129 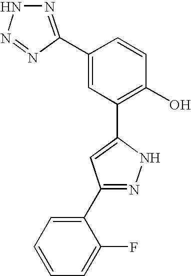
IA-130 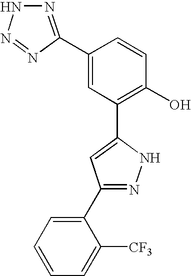
IA-131 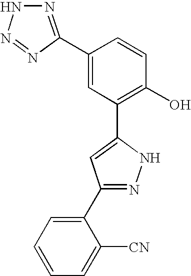
IA-132 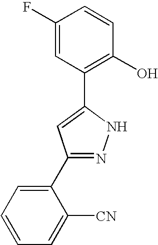
IA-133 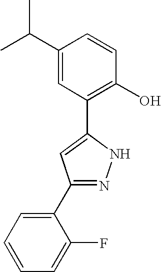
IA-134 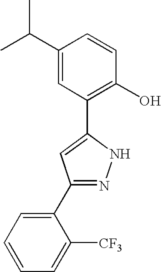
IA-135 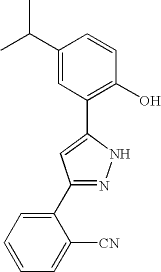
IA-136 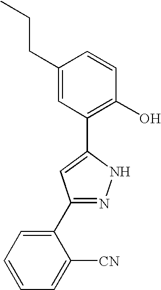
IA-137 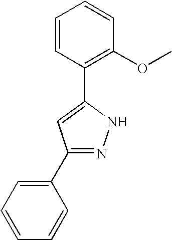
IA-138 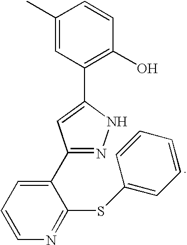
IA-139

TABLE 2 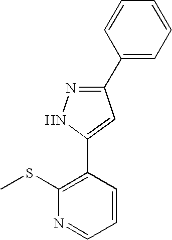
I-1 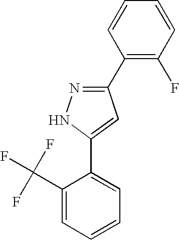
I-2 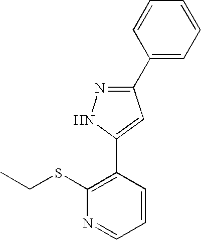
I-3 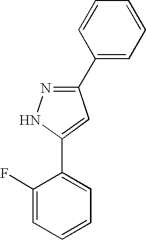
I-4 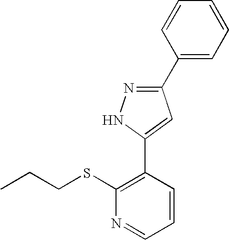
I-5 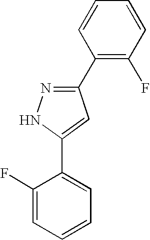
I-6 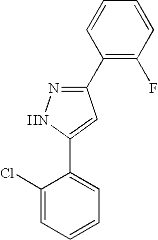
I-7 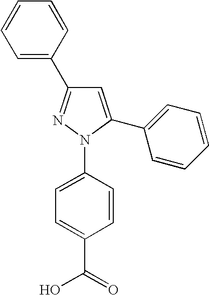
I-8 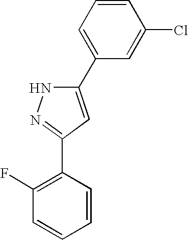
I-9 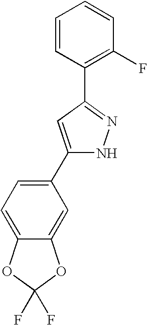
I-10 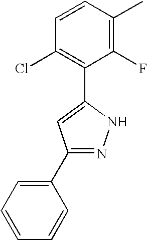
I-11 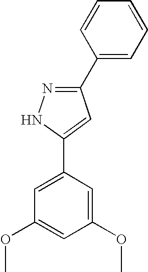
I-12 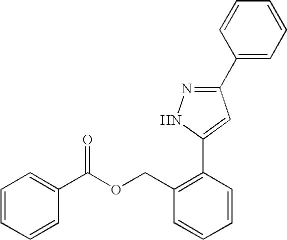
I-13 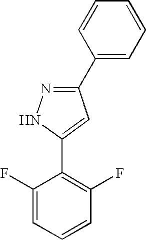
I-14 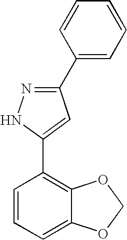
I-15 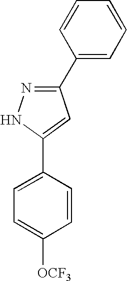
I-16 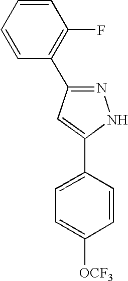
I-17 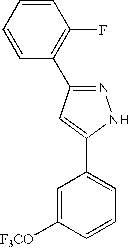
I-18 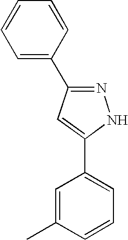
I-19 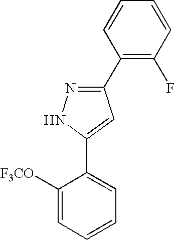
I-20 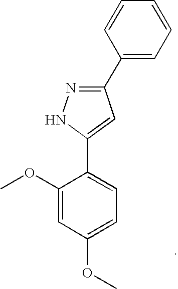
I-21

According to an alternative embodiment, preferred compounds of formula (I) are those that measurably increase the activity of an ABC-transporter or of a fragment thereof, and preferably CFTR activity.

According to another embodiment, preferred compounds of formula (I) are those that measurably decrease the activity of an ABC-transporter or of a fragment thereof.

One of skill in the art would be well aware of techniques and assays useful in measuring the increase or decrease of activity of an ABC-transporter or of a fragment thereof.

According to an alternative preferred embodiment, the present invention provides a method of modulating CFTR activity in a cell membrane of a mammal in need thereof, comprising the step of administering to said mammal a composition comprising a compound having the formula (I) as defined above. According to one embodiment, the compounds of the present invention potentiate the activity the CFTR in a cell membrane of a mammal in need thereof.

The preferred embodiments of compound of formula (I) useful in modulating the activity of CFTR include the preferred embodiments of formula (I) described above.

According to an alternative embodiment, the present invention provides a method of increasing the number of functional ABC transporters in a membrane of a cell, comprising the step of contacting said cell with a compound of formula (I). The term “functional ABC transporter” as used herein means an ABC transporter that is capable of transport activity.

According to a preferred embodiment, said functional ABC transporter is CFTR.

The preferred embodiments of compounds of formula (I) useful in increasing the number of functional ABC transporters include preferred embodiments of formula (I) as described above.

It will be apparent to one skilled in the art that some or all of the compounds of formula (I) may exist in two forms, e.g., as show below:

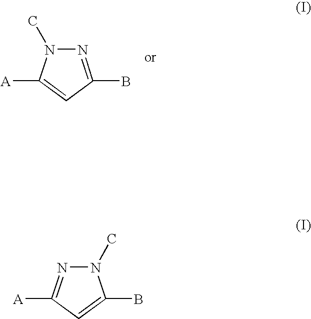

It is understood that the depiction of one form includes the depiction of the other and that all such isomeric forms, including tautomeric forms (when C is H) of the compounds are within the scope of this invention. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds that differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are within the scope of this invention. Such compounds are useful, for example, as analytical tools or probes in biological assays.

The present invention includes within its scope pharmaceutically acceptable prodrugs of the compounds of the present invention. A “pharmaceutically acceptable prodrug” means any pharmaceutically acceptable salt, ester, salt of an ester, or other derivative of a compound of the present invention which, upon administration to a recipient, is capable of providing (directly or indirectly) a compound of this invention or an active metabolite or residue thereof. Preferred prodrugs are those that increase the bioavailability of the compounds of this invention when such compounds are administered to a mammal or which enhance delivery of the parent compound to a biological compartment relative to the parent species.

The compounds of the present invention may be readily prepared using methods known in the art. One such synthetic route is illustrated in Scheme 1 below:

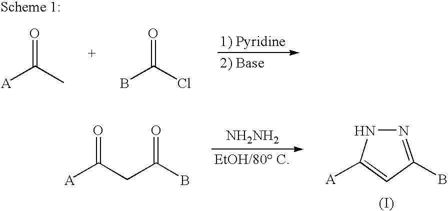

wherein A and B are as defined in formula (I). Compounds of formula (I) according to Scheme 1 are produced as a tautomeric mixture.

Scheme 1A below exemplifies the synthetic route of Scheme 1 for embodiments wherein A is 2-hydroxyphenyl, and B is phenyl. An example of a suitable base for this route is KOH.

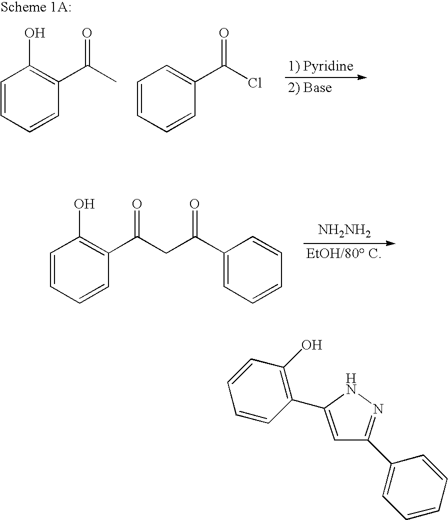

Scheme 2 below illustrates a yet another synthetic route that may be employed to produce compounds of formula (I).

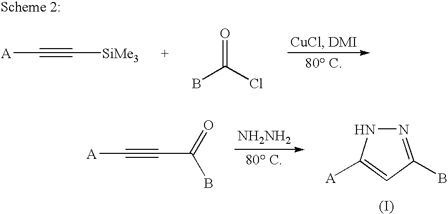

wherein A and B are as defined in formula (I). Compounds of formula (I) according to Scheme 2 are produced as a tautomeric mixture.

Scheme 2A below exemplifies the synthetic route of Scheme 1 for embodiments wherein A and B each is phenyl.

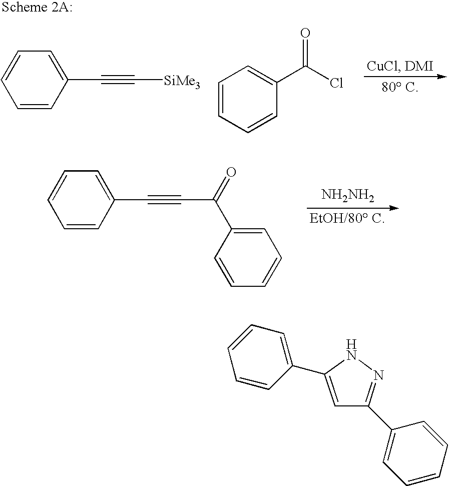

One of skill in the art will recognize that the above two synthetic routes are generic and can be readily exploited for any embodiment of compound formula (I).

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺ (C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the modulator can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated.”

According to an alternative embodiment, the present invention provides a method of treating a ABC transporter mediated disease in a mammal, comprising the step of administering to said mammal a composition comprising any one of compound of formula (I) to formula (X), or a preferred embodiment thereof as set forth above.

According to another embodiment, the ABC transporter mediated disease is selected from immunodeficiency disorder, inflammatory disease, allergic disease, autoimmune disease, destructive bone disorder, proliferative disorder, infectious disease or viral disease.

According to a another embodiment, the ABC transporter mediated disease is selected from Tangier's disease, stargardt disease 1, dry eye disease, age related macular dystrophy 2, retinintis pigmentosa, bare lymphocyte syndrome, PFIC-3, anemia, progressive intrahepatic cholestasis-2, Dublin-Johnson syndrome, Pseudoxanthoma elasticum, cystic fibrosis, familial persistent hyperinsulinemic hyproglycemia of infancy, adrenolecukodystrophy, sitosterolemia, chronic obstructive pulmonary disease, asthma, disseminated bronchiectasis, chronic pancreatitis, male infertility, emphysema, or pneumonia.

According to another embodiment, the ABC transporter mediated disease is secretory diarrhea, or polycystic kidney disease in a mammal.

According to an alternative embodiment, the present invention provides a method of treating cystic fibrosis or secretory diahrrea comprising the step of administering to said mammal a composition comprising a compound of the present invention. Preferably, said disease is cystic fibrosis.

According to another embodiment, the present invention provides a pharmaceutical composition comprising:

a compound of the present invention as described above;

a pharmaceutically acceptable carrier; and

an additional agent selected from a mucolytic agent, bronchodialator, an anti-biotic, an anti-infective agent, an anti-inflammatory agent, CFTR corrector, or a nutritional agent.

Embodiments of compounds formula (I) to formula (X) in the above pharmaceutical composition include the various embodiments of each of formula (I) through formula (X) described hereinabove.

According to another embodiment, the present invention provides a kit for use in measuring the activity of a ABC transporter or a fragment thereof in a biological sample in vitro or in vivo, comprising:

a composition comprising a compound of the present invention; and

instructions for:

contacting the composition with the biological sample;

measuring activity of said ABC transporter or a fragment thereof.

According to another embodiment, the kit is useful in measuring the activity of CFTR.

According to another embodiment, the activity of the ABC transporter is measured by measuring the transmembrane voltage potential.

Means for measuring the voltage potential across a membrane in the biological sample may employ any of the known methods in the art, such as optical membrane potential assay or other electrophysiological methods.

The optical membrane potential assay utilized voltage-sensitive FRET sensors described by Gonzalez and Tsien (See, Gonzalez, J. E. and R. Y. Tsien (1995) “Voltage sensing by fluorescence resonance energy transfer in single cells” Biophys J 69(4): 1272-80, and Gonzalez, J. E. and R. Y. Tsien (1997) “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC₂(3), and a fluorescent phospholipid, CC2-DMPE, which is attached to the outer leaflet of the plasma membrane and acts as a FRET donor. Changes in membrane potential (Vₘ) cause the negatively charged DiSBAC₂(3) to redistribute across the plasma membrane and the amount of energy transfer from CC2-DMPE changes accordingly. The changes in fluorescence emission can be monitored using VIPR™ II, which is an integrated liquid handler and fluorescent detector designed to conduct cell-based screens in 96- or 384-well microtiter plates.

Exemplary ABC transporters in the kit of the present invention include CFTR.

In order that the invention described herein may be more fully understood, the following examples are set forth. It should be understood that these examples are for illustrative purposes only and are not to be construed as limiting this invention in any manner.

### EXAMPLE 1

### Membrane Potential Optical Methods for Assaying ΔF508-CFTR Potentiation Properties of Compounds

The optical membrane potential assay utilized voltage-sensitive FRET sensors described by Gonzalez and Tsien (See, Gonzalez, J. E. and R. Y. Tsien (1995) “Voltage sensing by fluorescence resonance energy transfer in single cells”Biophys J 69(4): 1272-80, and Gonzalez, J. E. and R. Y. Tsien (1997) “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC₂(3), and a fluorescent phospholipid, CC2-DMPE, which is attached to the outer leaflet of the plasma membrane and acts as a FRET donor. Changes in membrane potential (Vₘ) cause the negatively charged DiSBAC₂(3) to redistribute across the plasma membrane and the amount of energy transfer from CC2-DMPE changes accordingly. The changes in fluorescence emission were monitored using VIPR™ II, which is an integrated liquid handler and fluorescent detector designed to conduct cell-based screens in 96- or 384-well microtiter plates.

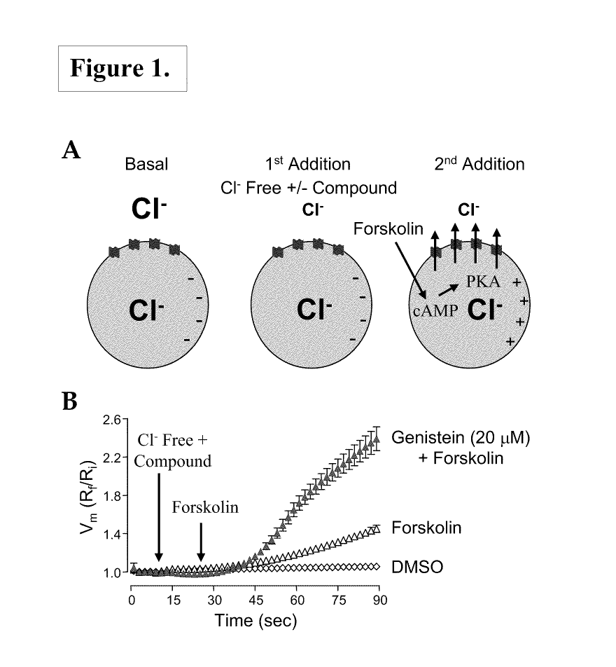

To identify potentiators of ΔF508-CFTR, a double-addition HTS assay format was developed (FIG. 1A). During the first addition, a Cl⁻-free medium with or without test compound was added to each well. After 22 sec, a second addition of Cl⁻-free medium containing 2-10 μM forskolin was added to activate ΔF508-CFTR. The extracellular Cl⁻ concentration following both additions was 28 mM, which promoted Cl⁻ efflux in response to ΔF508-CFTR activation and the resulting membrane depolarization was optically monitored using the FRET-based voltage-sensor dyes. The double-addition format has several advantages. First, it enables separation of compounds that act independently of forskolin-activated ΔF508-CFTR. Second, it allows compounds that act from the cytoplasmic surface of the channel to cross the plasma membrane and take effect. Lastly, fluorescent changes that arise from test compound addition alone can be identified. Under these assay conditions, the known CFTR potentiator, genistein, augmented the forskolin-induced membrane depolarization in NIH3T3 cells stably expressing ΔF508-CFTR (FIG. 1B). In the absence of forskolin addition, no response was observed in the presence (data not shown) or absence of genistein (FIG. 1B).

High-throughput assay format for identifying potentiators of ΔF508-CFTR stably expressed in NIH3T3 cells.

FIG. 1A: Double-addition assay format in which Cl⁻-free medium was added with or without the test compound prior to forskolin addition. The cells were incubated at 27° C. for 16-24 hr prior to use.
FIG. 1B: Membrane potential-response to forskolin following Cl⁻-free addition with or without Genistein (20 mM). No response was observed following addition of DMSO alone during the second addition.

### Solutions

Bath Solution #1: (in mM) NaCl 160, KCl 4.5, CaCl₂ 2, MgCl₂ 1, HEPES 10, pH 7.4 with NaOH.
Chloride-free bath solution: Chloride salts in Bath Solution #1 are substituted with gluconate salts.
CC2-DMPE: Prepared as a 10 mM stock solution in DMSO and stored at −20° C.
DiSBAC₂(3): Prepared as a 10 mM stock in DMSO and stored at −20° C.

### Cell Culture

NIH3T3 mouse fibroblasts stably expressing ΔF508-CFTR are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For all optical assays, the cells were seeded at 30,000/well in 384-well matrigel-coated plates and cultured for 2 hrs at 37° C. before culturing at 27° C. for 24 hrs.

In the optical assays, the known ΔF508-CFTR potentiator, genistein, potentiated the forskolin-induced response by 72.8±7.2% with an EC₅₀ of 19.2±1.9 μM (n=35). To compare the efficacy of the putative ΔF508-CFTR potentiators and genistein, the potentiation for each test compound was normalized to the peak genistein response in each plate. The data is normalized to % genistein response and the data is fitted using sigmoidal curve fit.

### Representative Example of VIPR Experiment

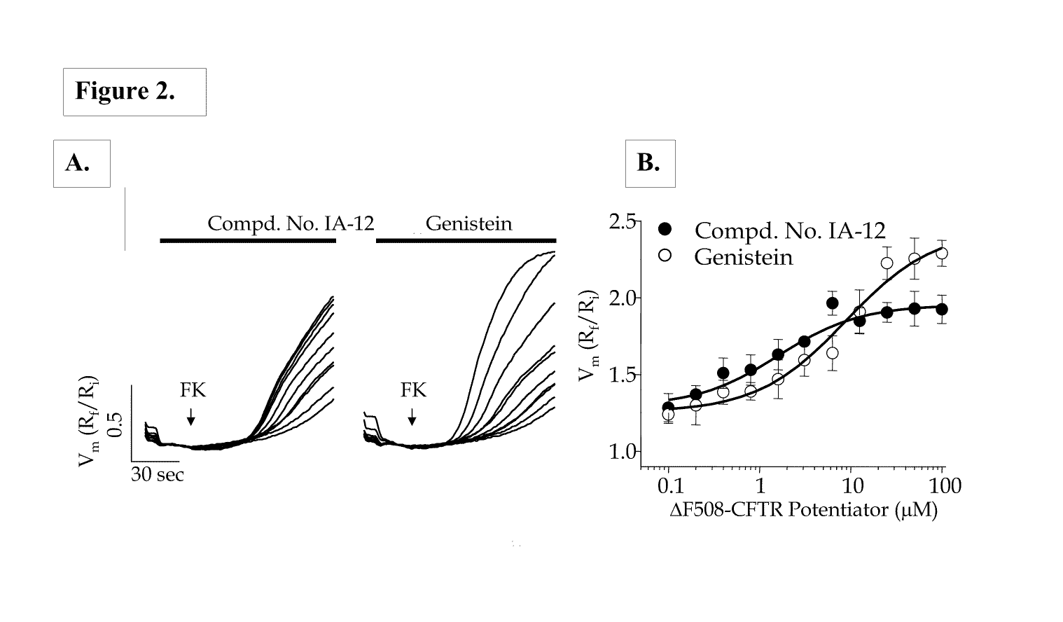

Dose response analysis of ΔF508-CFTR Potentiators.
FIG. 2A: Representative Vm curves of the response to 1 mM forskolin (FK) in the presence of the ΔF508-CFTR potentiators or genistein applied at concentrations from 100-0.1 μM.
FIG. 2B. Representative dose-response for the curves shown in FIG. 2A.

### EXAMPLE 2

### Electrophysiological Assays for Assaying ΔF508-CFTR Potentiation Properties of Compounds

### Ussing Chamber Assay

Ussing chamber experiments were performed on polarized epithelial cells expressing ΔF508-CFTR to further characterize the ΔF508-CFTR potentiators identified in the optical assays. FRT^{Δ508-CFTR} epithelial cells grown on Costar Snapwell cell culture inserts were mounted in an Ussing chamber (Physiologic Instruments, Inc., San Diego, Calif.), and the monolayers were continuously short-circuited using a Voltage-clamp System (Department of Bioengineering, University of Iowa, Iowa, and, Physiologic Instruments, Inc., San Diego, Calif.). Transepithelial resistance was measured by applying a 2-mV pulse. Under these conditions, the FRT epithelia demonstrated resistances of 4 KΩ/cm² or more. Typical protocol utilized a basolateral to apical membrane Cl⁻ concentration gradient. To set up this gradient, normal ringers was used on the basolateral membrane and was permeabilized with nystatin (360 μg/ml), whereas apical NaCl was replaced by equimolar sodium gluconate (titrated to pH 7.4 with NaOH) to give a large Cl⁻ concentration gradient across the epithelium. All experiments were performed 30 min after nystatin permeabilization. The solutions were maintained at 27° C. and bubbled with air. The electrode offset potential and fluid resistance were corrected using a cell-free insert. Under these conditions, the current reflects the flow of Cl⁻ through ΔF508-CFTR expressed in the apical membrane. The I_{SC} was digitally acquired using an MP100A-CE interface and AcqKnowledge software (version 3.2.6; BIOPAC Systems, Santa Barbara, Calif.). Forskolin (10 μM) and all test compounds were added to both sides of the cell culture inserts. The efficacy of the putative ΔF508-CFTR potentiators was compared to that of the known potentiator, genistein.

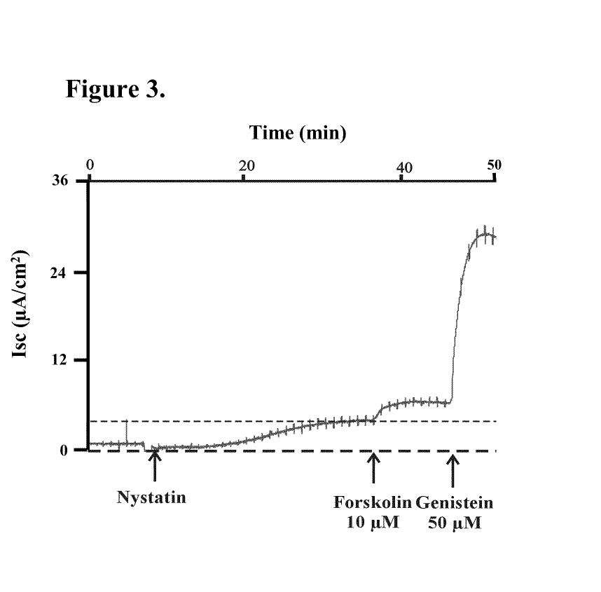

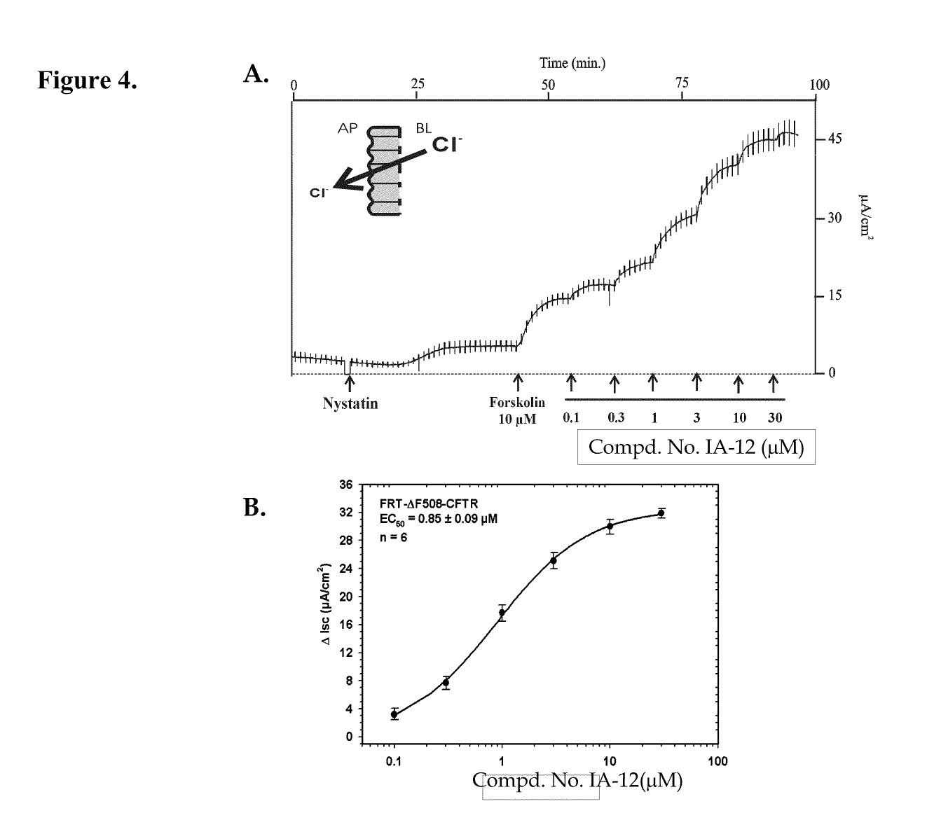

To confirm the activity of the putative potentiator compounds, their ability to potentiate the short-circuit current (I_{SC}) in FRT epithelia was determined using Ussing chamber measurement techniques. After nystatin permeabilization of the basolateral membrane, forskolin (10 μM) activated I_{SC} by 4.54±1.3 μA/cm² (n=30) (FIG. 3). Subsequent addition of genistein (50 μM) potentiated the I_{SC} to 21±1.5 μA/cm² (n=8). Application of genistein prior to forskolin addition did not stimulate I_{SC} (data not shown). No response to forskolin and genistein application was observed in parental FRT epithelia or FRT infected with the vector alone (data not shown). In the presence of 10 μM forskolin, Compd. No. IA-12 induced a dose-dependent increase in I_{SC} (FIGS. 4A and 4B) with an EC₅₀ of 0.85±0.09 μM (n=6). By comparison, genistein induced a dose-dependent response with an EC₅₀ of 21.2±0.49 μM (n=5) (data not shown). These results indicate that Compd. No. IA-12 potentiates the activity of forskolin-activated ΔF508-CFTR in polarized epithelia

### Solutions

Basolateral solution (in mM): NaCl (135), CaCl₂ (1.2), MgCl₂ (1.2), K₂HPO₄ (2.4), KHPO₄ (0.6), N-2-hydroxyethylpiperazine-N′-2-ethanesulfonic acid (HEPES) (10), and dextrose (10). The solution was titrated to pH 7.4 with NaOH.
Apical solution (in mM): Same as basolateral solution with NaCl replaced with Na Gluconate (135).

### Cell Culture

Fisher rat epithelial (FRT) cells expressing ΔF508-CFTR (FRT^{ΔF508-CFTR}) were used for Ussing chamber experiments for the putative ΔF508-CFTR potentiators identified from our optical assays. The cells were cultured on Costar Snapwell cell culture inserts and cultured for five days at 37° C. and 5% CO₂ in Coon's modified Ham's F-12 medium supplemented with 5% fetal calf serum, 100 U/ml penicillin, and 100 μg/ml streptomycin. Prior to use, the cells were incubated at 27° C. for 16-48 hrs to correct for the ΔF508-CFTR. Under our recording conditions, the FRT^{ΔF508-CFTR} epithelia exhibited a transepithelial resistance of 4KΩ/cm² or more.

Representative examples of Ussing Chamber experiment FIG. 3. Response to forskolin and genistein in ΔF508-CFTR expressing FRT epithelia. All cells were cultured at 27° C. for 16 hours prior to use. The response to forskolin and genistein application in ΔF508-CFTR-expressing FRT was inhibited by a CFTR antagonist.
FIG. 4A: Dose-dependent effect of Compd. No. IA-12 on nystatin permeabilized FRT^{ΔF508-CFTR} epithelia cultured at 27° C. overnight. Typical Isc current trace showing the concentration-dependent effects of Genistein and Compd. No. IA-12 on forskolin-activated Isc.
FIG. 4B: Dose-response curves of Compd. No. IA-12 for experiments shown in A. Values are the change in Isc, mean±SEM. for n=6. The EC₅₀ value for Compd. No. IA-12 is 0.85 μM

### Whole-Cell Recordings

The macroscopic ΔF508-CFTR current (I_{ΔF508-CFTR}) in temperature-corrected NIH3T3 cells stably expressing ΔF508-CFTR was monitored using the perforated-patch, whole-cell recording configuration. Briefly, voltage-clamp recordings of I_{ΔF508-CFTR} were performed at room temperature using an Axopatch 200B patch-clamp amplifier (Axon Instruments Inc., Foster City, Calif.). All recordings were acquired at a sampling frequency of 10 kHz and low-pass filtered at 1 kHz. Pipettes had a resistance of 5-6 MΩ when filled with the intracellular solution. Under the recording conditions, the calculated reversal potential for Cl⁻ (E_{Cl}) at room temperature was −28 mV. All recordings had a seal resistance >20 GΩ and a series resistance <15 MΩ. Pulse generation, data acquisition, and analysis were performed using a PC equipped with a Digidata 1320 A/D interface in conjunction with Clampex 8 (Axon Instruments Inc.). The bath contained <250 μl of saline and was continuously perifused at a rate of 2 ml/min using a gravity-driven perfusion system.

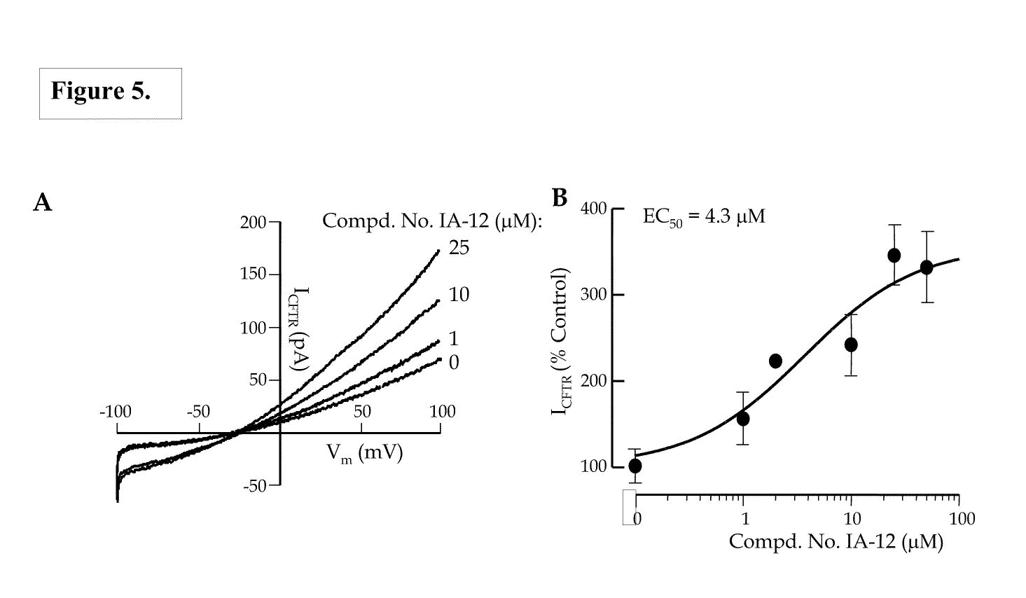

The ability of ΔF508-CFTR potentiator, Compd. No. IA-12, to increase the macroscopic ΔF508-CFTR Cl⁻ current (I_{ΔF508-CFTR}) in NIH3T3 cells stably expressing ΔF508-CFTR was investigated using perforated-patch-recording techniques. In four separate cells, Compd. No. IA-12 evoked a dose-dependent increase in I_{ΔF508-CFTR}, with an EC₅₀ of 4.3 μM (FIGS. 5A and B), which was similar to that obtained using the optical assay. In all cells examined, the reversal potential before and during Compd. No. IA-12 application was around −30 mV, which is the calculated E_{C1} (−28 mV). In addition, the effects of Compd. No. IA-12 were fully reversible within 2-min after its removal.

### Solutions

Intracellular solution (in mM): Cs-aspartate (90), CsCl (50), MgCl₂ (1), HEPES (10), and 240 μg/ml amphotericin-B (pH adjusted to 7.35 with CsOH).
Extracellular solution (in mM): N-methyl-D-glucamine (NMDG)-Cl (150), MgCl₂ (2), CaCl₂ (2), HEPES (10) (pH adjusted to 7.35 with HCl).

### Cell Culture

NIH3T3 mouse fibroblasts stably expressing ΔF508-CFTR are used for whole-cell recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For whole-cell recordings, 2,500-5,000 cells were seeded on poly-L-lysine-coated glass coverslips and cultured for 24-48 hrs at 27° C. before use.

### Representative Example of Whole-Cell Recording

Compd. No. IA-12 potentiates I_{ΔF508-CFTR} in NIH3T3 cells stably expressing ΔF508-CFTR.
FIG. 5A: Representative I_{ΔF508-CFTR}-voltage relationship for ΔF508-CFTR in the presence of 2 mM forskolin before and during application of 1, 10, and 25 mM Compd. No. IA-12.
FIG. 5B: Dose-dependent increase in the peak I_{ΔF508-CFTR} at 100 mV in response to increasing concentrations of Compd. No. IA-12. I_{ΔF508-CFTR} was normalized to the current amplitude in the presence of the vehicle control.

### Single-Channel Recordings

The single-channel activity of temperature-corrected ΔF508-CFTR stably expressed in NIH3T3 cells were observed using excised inside-out membrane patch. Briefly, voltage-clamp recordings of single-channel activity were performed at room temperature with an Axopatch 200B patch-clamp amplifier (Axon Instruments Inc.). All recordings were acquired at a sampling frequency of 10 kHz and low-pass filtered at 400 Hz. Patch pipettes were fabricated from Corning Kovar Sealing #7052 glass (World Precision Instruments, Inc., Sarasota, Fla.) and had a resistance of 5-8 MΩ when filled with the extracellular solution. After excision, ΔF508-CFTR was activated by adding 1 mM Mg-ATP, and 75 nM of the cAMP-dependent protein kinase, catalytic subunit (PKA; Promega Corp. Madison, Wis.). After channel activity stabilized, the patch was perifused using a gravity-driven microperifusion system. The inflow was placed adjacent to the patch, resulting in complete solution exchange within 1-2 sec. To maintain F508-CFTR activity during the rapid perifusion, the nonspecific phosphatase inhibitor F⁻ (10 mM NaF) was added to the bath solution. Under these recording conditions, channel activity remained constant throughout the duration of the patch recording (up to 60 min). Currents produced by positive charge moving from the intra- to extracellular solutions (anions moving in the opposite direction) are shown as positive currents. The pipette potential (Vₚ) was maintained at 80 mV.

Channel activity was analyzed from 8 membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Pₒ) were determined from 120 sec of channel activity. The Pₒ was determined using the Bio-Patch software or from the relationship Pₒ=I/i(N), where I=mean current, i=single-channel current amplitude, and N=number of active channels in patch.

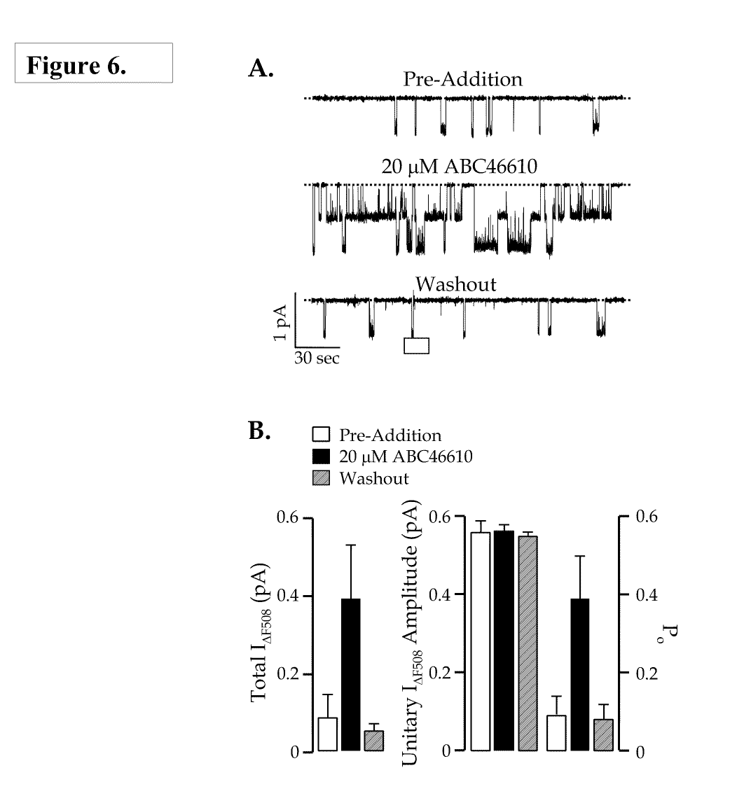

In four separate excised membrane patches, application of 20 μM Compd. No. IA-12 increased the total microscopic I_{F508}. FIG. 6 shows a representative I_{ΔF508-CFTR} trace before, during, and after application of 20 μM Compd. No. IA-12. Although application of Compd. No. IA-12 did not alter the single-channel amplitude, it did increase the number of functional channels observed in the membrane patch. In addition, Compd. No. IA-12 increased the Pₒ due to the increase in duration of the open bursts and decrease in the closed duration. Under identical recording conditions, the Pₒ of ΔF508-CFTR in the presence of 20 μM Compd. No. IA-12 was similar to that of the wild-type CFTR in the absence of agonist stimulation (data not shown). These results confirm that Compd. No. IA-12 acts directly on ΔF508-CFTR to increase its gating activity.

### Solutions

Extracellular solution (in mM): NMDG (150), aspartic acid (150), CaCl₂ (5), MgCl₂ (2), and HEPES (10) (pH adjusted to 7.35 with Tris base).
Intracellular solution (in mM): NMDG-Cl (150), MgCl₂ (2), EGTA (5), TES (10), and Tris base (14) (pH adjusted to 7.35 with HCl).

### Cell Culture

NIH3T3 mouse fibroblasts stably expressing ΔF508-CFTR are used for excised-membrane patch-clamp recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For whole-cell recordings, 2,500-5,000 cells were seeded on poly-L-lysine-coated glass coverslips and cultured for 24-48 hrs at 27° C. before use.

### Representative Example of Single-Channel Recordings

Direct action of Compd. No. IA-12 on ΔF508-CFTR in excised inside-out patches.
FIG. 6A: Representative single channel currents before, during and after application of 20 mM Compd. No. IA-12. FIG. 6B: Effects 20 mM Compd. No. IA-12 on total I_{ΔF508-CFTR}, unitary I_{ΔF508-CFTR} amplitude, and open probability (Po). The bath contained 1 mM ATP with 75 nM PKA to activate ΔF508-CFTR. All recordings were performed at room temperature and the membrane potential was clamped at −80 mV.

The relative modulating efficacy of the compounds of the present invention in comparison with genistein is recited below in Table 3.

“+++” means an efficacy range of >75% when compared to genistein.
“++” means an efficacy range of 35-75% when compared to genistein.
“+” means an efficacy range of <35% when compared to genistein.

TABLE 3

| Compd. No. | % Efficacy |
|---|---|
| IA-1 | ++ |
| IA-2 | ++ |
| IA-3 | ++ |
| IA-4 | ++ |
| IA-5 | +++ |
| IA-6 | +++ |
| IA-7 | ++ |
| IA-8 | ++ |
| IA-10 | ++ |
| IA-11 | ++ |
| IA-12 | +++ |
| IA-13 | +++ |
| IA-14 | +++ |
| IA-16 | ++ |
| IA-17 | ++ |
| IA-18 | ++ |
| IA-19 | ++ |
| IA-20 | +++ |
| IA-21 | ++ |
| IA-22 | ++ |
| IA-24 | ++ |
| IA-25 | ++ |
| IA-26 | +++ |
| IA-27 | ++ |
| IA-28 | +++ |
| IA-29 | ++ |
| IA-30 | ++ |
| IA-33 | ++ |
| IA-34 | + |
| IA-35 | +++ |
| IA-36 | ++ |
| IA-37 | ++ |
| IA-38 | ++ |
| IA-40 | ++ |
| IA-41 | ++ |
| IA-42 | +++ |
| IA-43 | +++ |
| IA-44 | ++ |
| IA-45 | +++ |
| IA-47 | +++ |
| IA-48 | + |
| IA-50 | ++ |
| IA-51 | ++ |
| IA-52 | +++ |
| IA-53 | ++ |
| IA-54 | ++ |
| IA-55 | ++ |
| IA-56 | ++ |
| IA-58 | ++ |
| IA-59 | ++ |
| IA-62 | ++ |
| IA-63 | + |
| IA-65 | ++ |
| IA-66 | ++ |
| IA-68 | ++ |
| IA-70 | ++ |
| IA-71 | ++ |
| IA-72 | ++ |
| IA-74 | ++ |
| IA-76 | ++ |
| IA-77 | ++ |
| IA-78 | ++ |
| IA-79 | + |
| IA-80 | ++ |
| IA-81 | ++ |
| IA-82 | ++ |
| IA-83 | ++ |
| IA-84 | ++ |
| IA-85 | ++ |
| IA-86 | ++ |
| IA-87 | ++ |
| IA-88 | ++ |
| IA-89 | ++ |
| IA-90 | ++ |
| IA-91 | ++ |
| IA-92 | ++ |
| IA-93 | ++ |
| IA-94 | ++ |
| IA-95 | ++ |
| IA-96 | ++ |
| IA-97 | ++ |
| IA-98 | ++ |
| IA-99 | ++ |
| IA-100 | ++ |
| IA-101 | ++ |
| IA-102 | ++ |
| IA-103 | + |
| IA-104 | ++ |
| IA-105 | ++ |
| IA-106 | ++ |
| IA-107 | ++ |
| IA-113 | + |
| IA-114 | ++ |
| IA-115 | + |
| I-1 | ++ |
| I-3 | ++ |
| I-4 | ++ |
| I-5 | ++ |
| I-6 | ++ |
| I-7 | ++ |
| I-9 | ++ |
| I-10 | ++ |
| I-15 | ++ |
| I-16 | ++ |
| I-17 | ++ |
| I-18 | ++ |
| I-19 | ++ |

### EXAMPLE 3

### 4-Methyl-2-(5-pyridin-3-yl-1H-pyrazol-3-yl)phenol

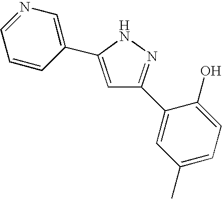

Pentafluorophenol trifluoroacetate (275 μL, 1.6 mmol) was added to a solution of nicotinic acid (197 mg, 1.6 mmol) in pyridine (2 mL) and the mixture was stirred at room temperature for 1 hour. 1-(2-Hydroxyphenyl)etanone (200 mg, 1.33 mmol) was added neat and the mixture was stirred at room temperature for an additional 2 hours followed by addition of KOH (224 mg, 4.0 mmol). After 12 hours at room temperature, hydrazine hydrate (131 μL, 2.7 mmol) was added and the reaction refluxed at 80° C. for 12 h. The mixture was filtered and purified by reverse phase HPLC (AcCN/H2O; 10 to 99%) to yield 96 mg of 4-Methyl-2-(5-pyridin-3-yl-1H-pyrazol-3-yl)phenol (24% yield). ¹H NMR (DMSO-d₆, 400 MHz): δ 2.27 (s, 3H), 6.84 (d, J=6.7 Hz, 1 H), 7.02 (d, J=6.7 Hz, 1H), 7.37 (s, 1H), 7.52 (s, 1H), 7.72 (m, 1H), 8.49 (m, 1H), 8.65 (m, 1H), 9.16 (s, 1H). EI-MS: m/z 252.0 (M+1).

### EXAMPLE 4

### 3-(2-fluorophenyl)-5-phenyl-1H-pyrazole

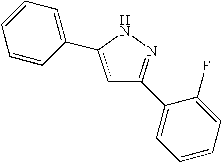

To a mixture of 1-phenyl-2-(trimethylsilyl)ethyne (174 mg, 1.0 mmol) and CuCl (20 mg, 0.2 mmol) in DMI (dimethylimidazolone) (0.5 mL) was added 2-fluorobenzoyl chloride (130.8 μL, 1.1 mmol) at room temperature. After stirring for 5 h at 80° C. the reaction was cooled to room temperature. Hydrazine hydrate was added (3.0 mmol, 145.5 μL) and the reaction was heated at 80° C. overnight. The reaction mixture was diluted with ethyl acetate (3 mL) and filtered through celite; the ethyl acetate was evaporated under reduced pressure and the resulting solution was filtered and purified by reverse phase HPLC purified by reverse phase HPLC (AcCN/H2O; 10 to 99%) to yield 166 mg of 3-(2-fluorophenyl)-5-phenyl-1H-pyrazole (70% yield). ¹H NMR (DMSO-d₆, 400 MHz): δ 7.07-8.0 (m, 10H). EI-MS: m/z 239.3.0 (M+1).

### EXAMPLE 5

### 4-Fluoro-2-[5(2-trifluoromethylphenyl)-1H-pyrazol-3-yl]phenol

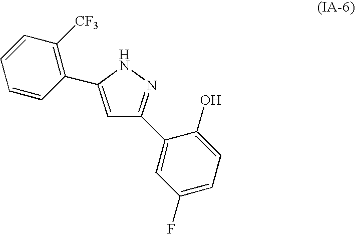

2-Trifluoromethyl-benzoyl chloride (572.7 μL, 3.89 mmol) was slowly added to a solution of 1-(5-fluoro-2-hydroxyphenyl)ethanone (500 mg, 3.24 mmol) in pyridine (2 mL) and the mixture was stirred at room temperature for 12 hours. KOH (545.4 mg, 9.72 mmol) was added and stirring was extended for an additional 12 h. The reaction was diluted with water (15 mL) and ethyl acetate (25 mL) and the aqueous layer was acidified to pH=1 with conc. HCl. The 2 layers were separated and the aqueous layer was extracted ethyl acetate (2×25 mL). The organic layers were combined, dried with MgSO₄, filtered and evaporated to yield a residue that was taken to the next step without further purification. The residue from previous step was dissolved in EtOH (10 mL), hydrazine hydrate (314.3 μL, 6.48 mmol) was added and the reaction was refluxed at 80° C. for 3 h. The solvent was evaporated and the crude residue purified by column chromatography with a gradient of ethyl acetate/hexanes 20 to 50% to yield 315 mg (30% yield, 2 steps) of 4-fluoro-2-[5(2-trifluoromethylphenyl)-1H-pyrazol-3-yl]phenol as a yellow crystalline material.

¹H NMR (CDCl₃, 400 MHz): δ 6.79 (s, 1H), 6.95-7.00 (m, 2H), 7.29 (dd, J=9.4 and 3.1 Hz, 1H), 7.58-7.71 (m, 3H), 7.85 (d, J=7.8 Hz, 1H). EI-MS: m/z 323.1 (M+1).

The following compounds were synthesized using the above methods.

| Compd. | LC MASS | | ¹H NMR | |
|---|---|---|---|---|
| No. | RT | M + H | (solvent) | ¹H NMR |
| IA-93 | 2.22 | 251.9 | | |
| IA-29 | 2.33 | 328.28 | | |
| IA-33 | 2.63 | 253.2 | | |
| IA-73 | 2.78 | 280.19 | | |
| IA-75 | 2.88 | 266.2 | | |
| IA-107 | 2.95 | 256.1 | | |
| IA-43 | 2.97 | 252.9 | | |
| IA-69 | 3 | 294 | | |
| IA-27 | 3.02 | 252.18 | DMSO | 2.27 (s, 3H), |
| | | | | 6.84 (d, J = 6.7 Hz, 1H), |
| | | | | 7.02 (d, J = 6.7 Hz, |
| | | | | 1H), 7.37 (s, |
| | | | | 1H), 7.52 (s, 1H), |
| | | | | 7.72 (m, 1H), |
| | | | | 8.49 (m, 1H), 8.65 (m, |
| | | | | 1H), 9.16 (s, 1H). |
| IA-40 | 3.03 | 308.3 | | |
| IA-76 | 3.04 | 395.4 | | |
| I-2 | 3.06 | 307.16 | | |
| IA-54 | 3.08 | 305.21 | | |
| I-7 | 3.13 | 273.6 | | |
| I-17 | 3.14 | 323.16 | | |
| IA-97 | 3.14 | 282.1 | | |
| I-1 | 3.14 | 297.1 | | |
| I-20 | 3.17 | 323.16 | | |
| IA-34 | 3.18 | 266.9 | | |
| I-18 | 3.19 | 323.16 | | |
| I-10 | 3.2 | 319.15 | | |
| IA-104 | 3.3 | 268.9 | | |
| I-7 | 3.32 | 273.6 | | |
| IA-11 | 3.33 | 267.1 | | |
| IA-108 | 3.38 | 254.9 | | |
| I-6 | 3.39 | 257.15 | | |
| IA-105 | 3.4 | 318.9 | | |
| IA-109 | 3.4 | 267.1 | | |
| IA-55 | 3.41 | 301.1 | | |
| IA-16 | 3.41 | 335.3 | | |
| IA-2 | 3.42 | 285.1 | | |
| IA-102 | 3.45 | 296.3 | | |
| IA-87 | 3.45 | 295.9 | | |
| IA-81 | 3.45 | 254.9 | | |
| IA-25 | 3.45 | 295.1 | | |
| IA-42 | 3.47 | 323.18 | | |
| IA-100 | 3.47 | 281.1 | | |
| IA-28 | 3.47 | 256.9 | | |
| IA-92 | 3.48 | 327.15 | | |
| IA-22 | 3.48 | 281.1 | | |
| IA-9 | 3.49 | 265.2 | | |
| IA-32 | 3.5 | 297.1 | | |
| IA-24 | 3.5 | 250.9 | | |
| IA-71 | 3.51 | 257.24 | | |
| IA-63 | 3.52 | 268.9 | | |
| IA-47 | 3.53 | 368.9 | | |
| IA-62 | 3.54 | 281.1 | | |
| IA-106 | 3.55 | 268.9 | | |
| IA-49 | 3.55 | 271.1 | | |
| IA-110 | 3.56 | 271.1 | | |
| I-15 | 3.57 | 265.18 | | |
| IA-20 | 3.58 | 300.16 | | |
| IA-44 | 3.58 | 286.9 | | |
| IA-46 | 3.64 | 264.9 | | |
| IA-48 | 3.66 | 285.1 | | |
| I-8 | 3.67 | 341.1 | | |
| IA-91 | 3.67 | 484.3 | | |
| IA-58 | 3.67 | 285.1 | | |
| IA-59 | 3.67 | 264.9 | | |
| IA-21 | 3.67 | 255.2 | DMSO | 6.95 (dd, J = 8.5, |
| | | | | 4.8 Hz, 1H), |
| | | | | 7.03 (td, J = 11.4, 3 Hz, |
| | | | | 1H), 7.33-7.40 (m, |
| | | | | 2H), 7.47-7.51 (m, |
| | | | | 2H), 7.60 (d, J = 13.6 Hz, |
| | | | | 1H), 7.83 (d, |
| | | | | J = 7.3 Hz, 2H). |
| IA-15 | 3.69 | 301.1 | | |
| IA-88 | 3.7 | 288.9 | | |
| IA-65 | 3.7 | 430.1 | | |
| IA-31 | 3.7 | 385.3 | | |
| IA-21 | 3.71 | 281.22 | | |
| IA-94 | 3.73 | 281.1 | | |
| IA-82 | 3.74 | 264.9 | | |
| I-12 | 3.77 | 281.22 | | |
| IA-23 | 3.77 | 265.22 | | |
| IA-111 | 3.78 | 318.9 | | |
| IA-17 | 3.78 | 333 | | |
| IA-79 | 3.79 | 333.3 | | |
| IA-53 | 3.79 | 279.3 | | |
| IA-51 | 3.8 | 303.1 | | |
| IA-37 | 3.82 | 268.25 | | |
| IA-99 | 3.85 | 299.21 | | |
| IA-78 | 3.85 | 273.15 | | |
| I-19 | 3.86 | 234.8 | | |
| IA-83 | 3.87 | 414.3 | | |
| IA-57 | 3.88 | 350.9 | | |
| IA-90 | 3.91 | 339.1 | | |
| IA-74 | 3.93 | 333.1 | | |
| IA-85 | 3.96 | 293.1 | | |
| IA-10 | 3.96 | 357.1 | | |
| IA-96 | 3.98 | 292.9 | | |
| IA-19 | 4 | 271.61 | DMSO | 6.95 (m, 1H), |
| | | | | 7.23 (m, 1H), |
| | | | | 7.42-7.51 (m, 4H), |
| | | | | 7.81-7.87 (m, 3H) |
| IA-84 | 4.04 | 343.1 | | |
| IA-37 | 4.05 | 284.2 | | |
| IA-4 | 4.06 | 323.3 | | |
| IA-45 | 4.08 | 335.2 | | |
| I-16 | 4.08 | 305.17 | | |
| I-3 | 4.09 | 282.28 | | |
| IA-50 | 4.1 | 311.1 | | |
| IA-56 | 4.11 | 301.4 | | |
| IA-7 | 4.11 | 385.1 | | |
| IA-12 | 4.12 | 254.3 | | |
| I-14 | 4.16 | 256.8 | | |
| IA-112 | 4.17 | 267.05 | | |
| | 4.19 | 237.3 | | |
| | 4.2 | 295.4 | | |
| IA-77 | 4.2 | 360.28 | | |
| IA-36 | 4.27 | 265.22 | | |
| | 4.37 | 360.35 | | |
| IA-101 | 4.39 | 282.21 | | |
| IA-26 | 4.41 | 278.8 | | |
| | 4.43 | 287.63 | | |
| IA-14 | 4.49 | 278.8 | | |
| IA-13 | 4.56 | 355.3 | | |
| IA-86 | 4.62 | 394.79 | | |
| IA-41 | 4.63 | 319.19 | | |
| IA-89 | 4.81 | 353.2 | | |
| I-5 | 4.81 | 296.3 | | |
| IA-70 | 5.32 | 255.16 | | |
| IA-98 | 4.13 | 323.2 | | |

1. A method of modulating ABC transporter activity comprising the step of contacting said ABC transporter with a compound of formula (I): 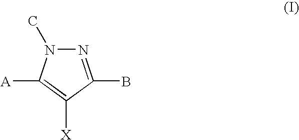
or a pharmaceutically acceptable salt thereof;
wherein:
A and B are independently selected from aryl, heterocyclic, heteroaryl, or cycloaliphatic ring;
C is H, aryl, heterocyclic, heteroaryl, cycloaliphatic, aliphatic, C(O)R², C(O)R³, C(O)NH₂, C(O)NHR², C(O)NHR³, C(O)N(R²)₂, C(O)N(R³)₂;
X is H, (CH₂)ₙ—Y, R², R³, R⁴, R⁵, or R⁶;
wherein each of A, B, C, and X optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶ or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CHF₂, CH₂F, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group.

2. The method according to claim 1, wherein each of C and X is H.

3. The method according to claim 2, wherein A and B are independently optionally substituted aryl or heteroaryl.

4. The method according to claim 3, wherein A and B are independently selected from optionally substituted phenyl, pyrazolyl, pyridyl, thiazolyl, oxazolyl, thiophenyl, or furanyl.

5. The method according to claim 1, wherein B is selected from optionally substituted ring systems:

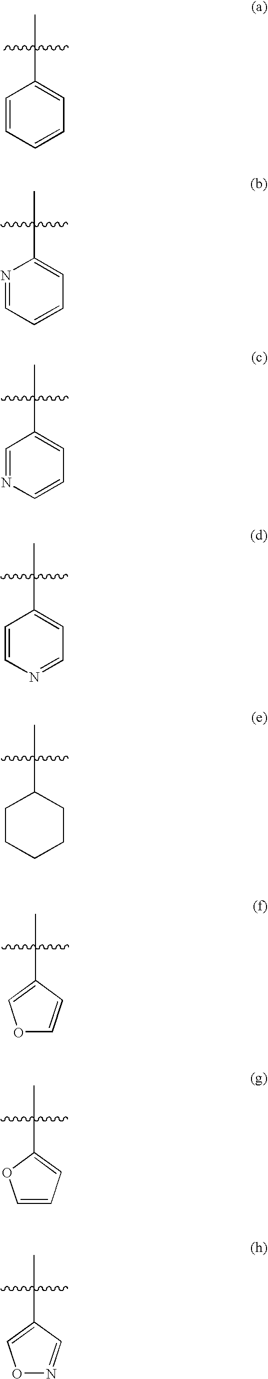

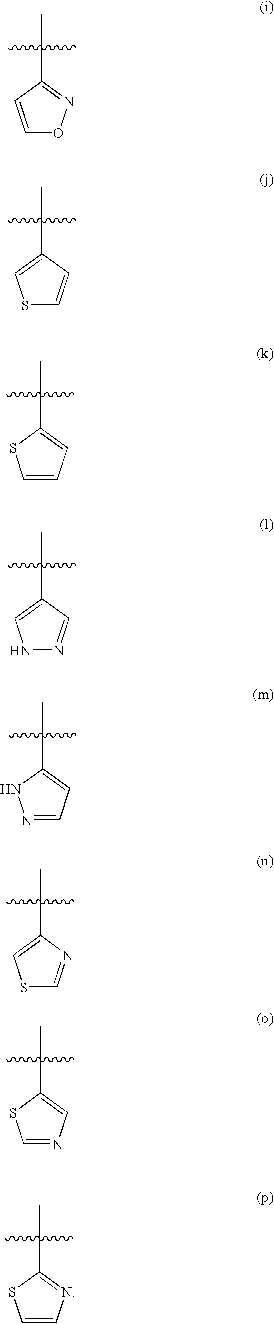

6. The method according to claim 1, wherein said formula (IA): 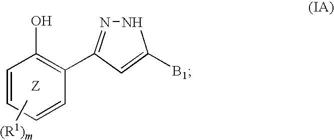
wherein:
m is 0 to 3;
B₁ is selected from: 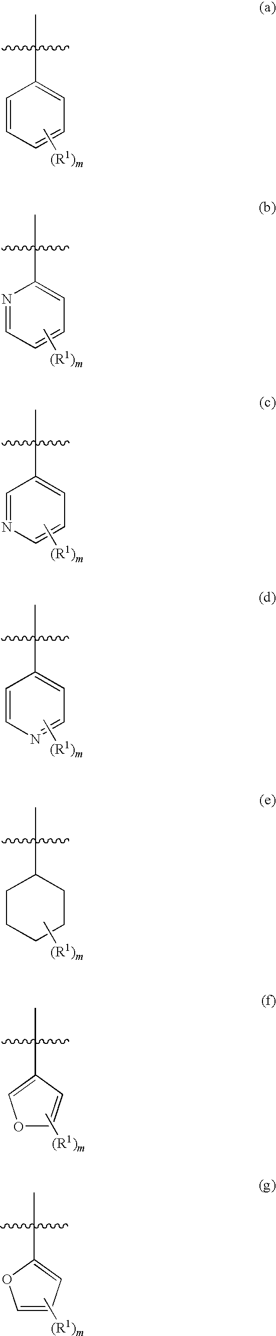 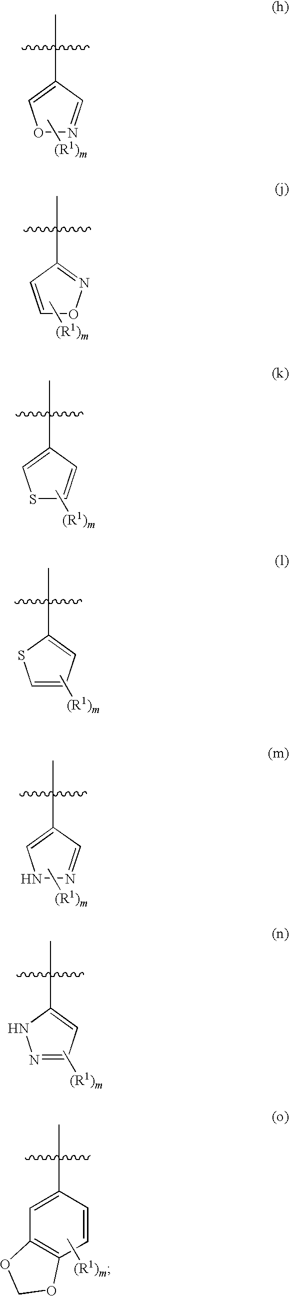
wherein B₁ and ring Z are substituted with up to 2 substituents selected from R², R³, or R⁴.

7. The method according to any one of claims 6, wherein R¹ is selected from halo, CF₃, NH₂, NH(C1-C6 alkyl), NHC(O)CH₃, OH, O(C1-C6 alkyl), OPh, O-benzyl, S—(C1-C6 alkyl), C1-C6 alkyl, NO₂, CN, methylenedioxy, ethylenedixoy, SO₂NH(C1-C6 alkyl), or SO₂N(C1-C6 alkyl)₂.

8. The method according to claim 1, wherein said compound is selected compounds IA-1 to IA-139 in Table 1 compound I-1 to I-21 in Table 2.

9. The method according to claim 1, wherein said compound has formula (II): 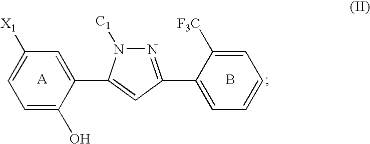
or a pharmaceutically acceptable salt thereof, wherein:
C₁ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
X₁ is selected from halo, R², CF₃, CN, COOH, COOR, C(O)R, C(O)NH₂, C(O)NHR, or C(O)N(R)₂;
each R is independently R² or R³;
wherein each of ring B, optionally including X₁ and OH, and C₁ optionally comprises up to 4 substituents, and ring A optionally comprises up to 3 substituents, wherein said substituents are independently selected from R¹, R², R³, R⁴, or R⁵;
R¹ is R⁶ or (CH₂)ₙ—Y;
n is 0, 1 or 2;
Y is halo, CN, NO₂, CF₃, CHF₂, CH₂F,
OCF₃, OH, SCHF₂, SR⁶, S(O)R⁶, SO₂R⁶, NH₂, NHR⁶, N(R⁶)₂, NR⁶R⁸, COOH, COOR⁶ or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CHF₂, CH₂F,
CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic), or O-aliphatic; and
R⁸ is an amino protecting group.

10. The method according to claim 9, wherein C₁ is H.

11. The method according to claim 10, wherein
X₁ is selected from (C1-C4)-aliphatic, or C(O)—NH₂.

12. The method according to claim 1, wherein said compound has formula provides a compound having formula (III): 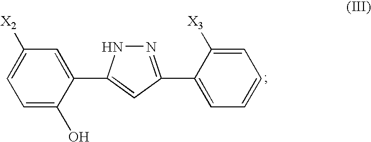
or a pharmaceutically acceptable salt thereof, wherein:
X₂ is selected from halo, R², CF₃, CN, COOH, COOR², COOR³, C(O)R², C(O)R³, C(O)NH₂, C(O)NHR, or C(O)NR²;
X₃ is selected from H, halo, CF₃, or NO₂;
each R is independently R² or R³;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶ or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CHF₂, CH₂F,
CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group;
provided that:
(i) when X₃ is H, then X₂ is not methyl, chloro, or bromo;
(ii) when X₂ is chloro, then X₃ is not fluoro, chloro, or nitro;
(iii) when X₂ is methyl, then X₃ is not nitro or chloro.

13. The method according to claim 12, said compound has one or more of the features selected from the group:
(a) X₃ is halo, CF₃, or NO₂; and
(b) X₂ is halo, CF₃, methyl, ethyl, propyl, or CONH₂.

14. The method according to claim 1, wherein said compound has formula (IV): 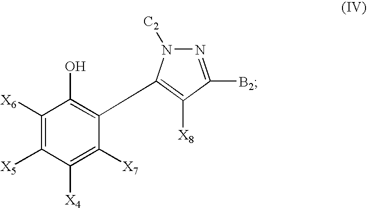
or a pharmaceutically acceptable salt thereof;
wherein:
B₂ is selected from: 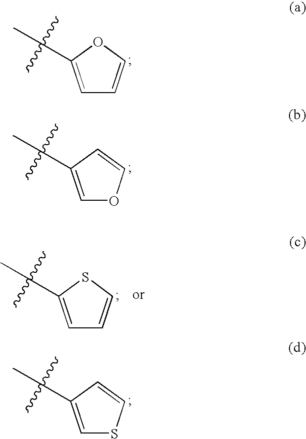
C₂ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
each of X₄, X₅, X₆, X₇, and X₈ is selected from (CH₂)ₙ—Y, R², R³, R⁴, R⁵ or R⁶;
wherein each of B₂ and C₂ optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic), or O-aliphatic; and
R⁸ is an amino protecting group;
provided that:
(i) when B₂ is structure (a), X₅, X₆, and C₂ are H, then X₄ is not H, Cl, CH₃, or OCH₃;
(ii) when B₂ is structure (c), X₅, X₆, and C₂ is H, then X₄ is not H or CH₃;
(iii) when B₂ is structure (a), X₄ is Cl or CH₃, X₅ and C₂ are H, then X₆ is not NO₂, Cl, or Br;
(iv) when B₂ is structure (a), X₄ is Cl, X₅ and X₆ are H, then C₂ is not Ph, —C(O)CH₃, —C(O)Ph, or —C(O)NHPh;
(v) when B₂ is structure (a), X₄ is CH₃, X₅ and X₆ is H; then C₂ is not Ph;
(vi) when B₂ is structure (a), X₄, X₅, and X₆ is H, then C₂ is not CH₃, C(O)CH₃, or —C(O)—NHPh;
(vii) when B₂ is structure (c), X₄, X₅, and X₆ is H, then C₂ is not CH₃ or C(O)CH₃;
(viii) when B₂ is structure (a), X₄ is Cl, X₅ is H, X₆ is NO₂ or Br, then X₂ is not Ph, C(O)CH₃, or C(O)Ph.

15. The method according to claim 14, wherein B₂ is optionally substituted ring

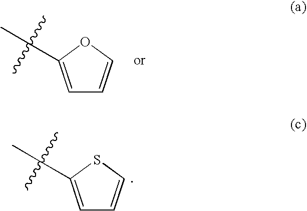

16. The method according to claim 15, wherein X₈ and C₂ are H.

17. The method according to claim 16, wherein compounds of formula (IV) have one or more of the features selected from the group:
(a) B₂ is:
5-(3′-trifluoromethylphenyl)-furan-2-yl;
5-trifluoromethyl-2-methyl-furan-3-yl;
5-t-butyl-2-methyl-furan-3-yl;
5-methyl-2-trifluoromethyl-furan-3-yl; or
5-(4′-methylsulfonylphenyl)-furan-2-yl;
(b) C₂ is H or phenyl;
(c) X₄ is halo, (C1-C4)alkyl, CF₃, CN, or NO₂;
(d) X₅, X₆, and X₇ are H; and
(e) X₈ is H.

18. The method according to claim 16, wherein X₄, X₅, X₆, and X₇, taken together with the hydroxyphenyl group, is selected from 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl 2-hydroxy-5-methylsulfonylphenyl, or 2-hydroxy-5-amidophenyl.

19. The method according to claim 1, wherein said compound has formula (V): 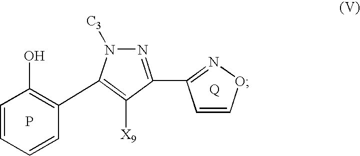
or a pharmaceutically acceptable salt thereof;
wherein:
C₃ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
X₉ is selected from (CH₂)ₙ—Y, R², R³, R⁴, R⁵ or R⁶;
wherein each of ring P, optionally including the hydroxyl group, and ring Q optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CHF₂, CH₂F,
CF₃, OCF₃, OH, SCHF₂, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group.

20. The method according to claim 19, wherein X₉ and C₃ are H.

21. The method according to claim 20, wherein, said compound has one or more of the features selected from the group:
(a) C₃ is H or phenyl;
(b) ring Q is isoxazol-3-yl or 5-methyl-isoxazol-3-yl;
(c) X₉ is H; and
(d) ring P together with the hydroxy substituent is selected from:
2-hydroxy-5-methoxyphenyl,
2-hydroxy-5-methylphenyl,
2-hydroxy-5-fluorophenyl,
2-hydroxy-5-ethylphenyl,
2-hydroxy-5-propylphenyl,
2-hydroxy-5-chlorophenyl,
2-hydroxy-5-isopropylphenyl,
2-hydroxy-5-tetrazol-2H-3-ylphenyl,
2-hydroxy-5-bromophenyl,
2-hydroxy-5-methylsulfonylphenyl, or
2-hydroxy-5-amidophenyl.

22. The method according to claim 1, wherein said compound has formula (VI): 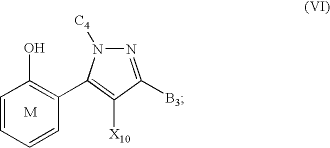
or a pharmaceutically acceptable salt thereof;
wherein:
B₃ is selected from: 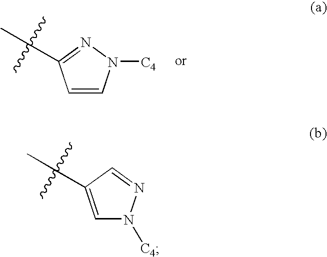
C₄ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
X₁₀ is selected from (CH₂)ₙ—Y, R², R³, R⁴, R⁵ or R⁶;
wherein each of ring M, optionally including the hydroxyl group, C₄, and B₃ optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)², NR⁶R⁸, COOH, COOR⁶, or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group.

23. The method according to claim 22, wherein X₁₀ and C₄ are H.

24. The method according to claim 23, wherein B₃ is optionally substituted ring

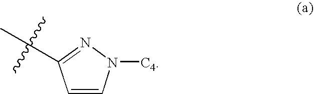

25. The method according to claim 24, wherein, ring M, together with the 2-hydroxy group, is selected from 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl, 2-hydroxy-5-methyl sulfonylphenyl, or 2-hydroxy-5-amidophenyl.

26. The method according to claim 1, wherein said compound has formula (VII): 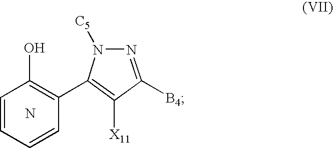
or a pharmaceutically acceptable salt thereof;
wherein:
B₄ is selected from: 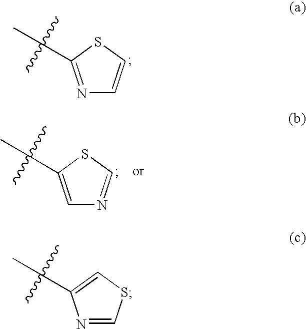
C₅ is H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
X₁₁ is selected from (CH₂)ₙ—Y, R², R³, R⁴, R⁵ or R⁶;
wherein each of ring N, optionally including the hydroxyl group, C₅, and B₄ optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group;
provided that:
(a) when C₅ is H, X₁₁ is H, ring N is 2-hydroxy-4-methoxyphenyl, then B₄ is not 2-methylthiazol-4-yl;
(b) when C₅ is H, X₁₁ is H, ring N is 2-hydroxy-4,5-dimethylphenyl, then B₄ is not 2-methylthiazol-4-yl.

27. The method according to claim 26, wherein X₁₁ and C₅ are H.

28. The method according to claim 27, wherein B₄ is optionally substituted

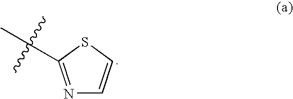

29. The method according to claim 27, wherein ring N, together with the 2-hydroxy group, is selected from 2-hydroxy-5-methoxyphenyl, 2-hydroxy-5-methylphenyl, 2-hydroxy-5-fluorophenyl, 2-hydroxy-5-ethylphenyl, 2-hydroxy-5-propylphenyl, 2-hydroxy-5-chlorophenyl, 2-hydroxy-5-isopropylphenyl, 2-hydroxy-5-tetrazol-2H-3-ylphenyl, 2-hydroxy-5-bromophenyl, 2-hydroxy-5-methylsulfonylphenyl, 2-hydroxy-5-amidophenyl, 2-hydroxy-6-methoxyphenyl, 2-hydroxy-4,6-dimethylphenyl, 2-hydroxy-4,5-dimethylphenyl, 2-hydroxy-4-methylphenyl, or 2-hydroxy-4-fluorophenyl.

30. The method according to claim 1, wherein said compound has formula (VIII): 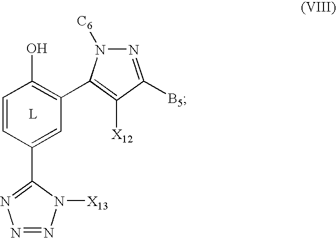
or a pharmaceutically acceptable salt thereof, wherein:
B₅ is optionally substituted aryl, heteroaryl, cycloaliphatic, or heterocyclyl;
C₆ and X₁₃ each is independently selected from H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
X₁₂ is selected from (CH₂)ₙ—Y, R², R³, R⁴, R⁵ or R⁶;
wherein each of ring L, including the hydroxyl group, C₆, and B₅ optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group.

31. The method according to claim 30, wherein X₁₂, X₁₃, and C₆ is phenyl.

32. The method according claim 31, wherein B₅ is optionally substituted phenyl.

33. The method according to claim 31, wherein B₅ is selected from 2-methoxyphenyl, 3-methoxyphenyl, 4-methoxyphenyl, 2,4-dimethoxy-phenyl, 3,4-dimethoxy-phenyl, 3,5-dimethoxy-phenyl, 4-hydroxyphenyl, 3-hydroxyphenyl, 2-hydroxyphenyl, 2-chloro-phenyl, 4-chloro-phenyl, 2,6-dichloro-phenyl, 4-fluoro-phenyl, 3-fluoro-phenyl, 2-fluoro-phenyl, 3,4-difluoro-phenyl, 2,6-difluoro-phenyl, phenyl, 4-butoxy-phenyl, 2-ethoxy-phenyl, 2-nitro-phenyl, 3-nitro-phenyl, 4-nitro-phenyl, 2-trifluoromethoxy-phenyl, 3-trifluoromethoxy-phenyl, 4-trifluoromethoxy-phenyl, 2-trifluoromethyl-phenyl, 4-trifluoromethyl-phenyl, 5-(3-trifluoromethyl-phenyl)-furan-2-yl, 4-benzyloxy-phenyl, 3-methyl-4-trifluoromethyl-phenyl, 2-methyl-phenyl, 3-methyl-phenyl, 4-methyl-phenyl, benzo[1,3]dioxol-5-yl, pyridin-3-yl, pyridin-4-yl, thiophen-2-yl, 2-pyridin-4-yl-phenyl, 2-benzonitrile, 1-phenyl-4-trifluoromethyl-1H-pyrazolyl, 4-bromophenyl, 2-methylsulfanyl-pyridin-3-yl, 2-ethylsulfanyl-pyridin-3-yl, 2-propylsulfanyl-pyridin-3-yl, 2-benzoic acid methyl ester, N-3-phenyl-acetamide, 2-methyl-5-trifluoromethyl-furan-3-yl, 5-Methyl-2-trifluoromethyl-furan-3-yl), 5-tent-butyl-2-methyl-furan-3-yl, 3-chloro-4-fluoro-phenyl, 2,3-dimethyl-phenyl, 2,6-difluoro-3-methyl-phenyl, 2-(4-nitro-phenyl)-5-trifluoromethyl-pyrazolyl-5-yl, 4-tert-butyl-phenyl, 4-dimethylamino-phenyl, cyclohexyl, 4-methoxy-3-trifluoromethyl-phenyl; 2-methyl-3-trifluoromethyl-phenyl, 2-amino-phenyl, 5-(4-methanesulfonyl-phenyl)furan-2-yl, 2-phenoxy-pyridin-3-yl; 2-difluoromethylsulfanyl-phenyl, N,N-diethyl-4-benzenesulfonamide, 2-phenoxy-phenyl, 2,4,6-trimethyl-phenyl, 2-(4-chloro-phenylsulfanyl)-pyridin-3-yl], 5-chloro-2-trifluoromethyl-phenyl, 5-methyl-2-trifluoromethyl-furan-3-yl, 5-(2,3-dihydro-benzofuran-6-yl)-4-methyl-thiazol-2-yl, 2-fluoro-4-trifluoromethyl-phenyl, 2-fluoro-4-methoxy-phenyl, 2-ethoxy-pyridin-3-yl, 5-methyl-isoxazol-3-yl), 4-benzoic acid, 2,2-difluoro-benzo[1,3]dioxol-5-yl, benzoic acid 2-benzyl ester,
5-benzo[1,3]dioxol-4-yl.

34. The method according to claim 1, wherein said compound has formula (IX): 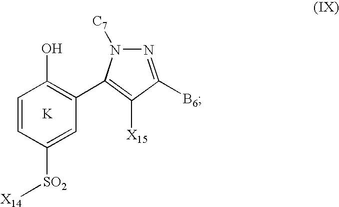
or a pharmaceutically acceptable salt thereof, wherein:
B₆ is phenyl;
C₇ is selected from H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
X₁₄ is R², R³, NHR², NHR³, NR²R³, N(R²)₂;
X₁₅ is selected from (CH₂)ₙ—Y, R², R³, R⁴, R⁵ or R⁶;
wherein each of ring K, optionally including the hydroxyl group, C₇, and B₆ optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)ORS, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶), NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group.

35. The method according to claim 34, wherein X₁₅ and C₇ are phenyl.

36. The method according to claim 35, wherein X₁₄ is selected from optionally substituted (C1-C6)aliphatic, aryl, NH(C1-C6)aliphatic, NH(aryl), or NH₂. Preferred X₁₄ include optionally substituted (C1-C4)-alkyl, phenyl, NH[(C1-C4)-alkyl], NH(phenyl), or NH₂.

37. The method according to claim 36, wherein B₆ is selected from 2-methoxyphenyl, 3-methoxyphenyl, 4-methoxyphenyl, 2,4-dimethoxy-phenyl, 3,4-dimethoxy-phenyl, 3,5-dimethoxy-phenyl, 4-hydroxyphenyl, 3-hydroxyphenyl, 2-hydroxyphenyl, 2-chloro-phenyl, 4-chloro-phenyl, 2,6-dichloro-phenyl, 4-fluoro-phenyl, 3-fluoro-phenyl, 2-fluoro-phenyl, 3,4-difluoro-phenyl, 2,6-difluoro-phenyl, phenyl, 4-butoxy-phenyl, 2-ethoxy-phenyl, 2-nitro-phenyl, 3-nitro-phenyl, 4-nitro-phenyl, 2-trifluoromethoxy-phenyl, 3-trifluoromethoxy-phenyl, 4-trifluoromethoxy-phenyl, 2-trifluoromethyl-phenyl, 4-trifluoromethyl-phenyl, 5-(3-trifluoromethyl-phenyl)-furan-2-yl, 4-benzyloxy-phenyl, 3-methyl-4-trifluoromethyl-phenyl, 2-methyl-phenyl, 3-methyl-phenyl, 4-methyl-phenyl, benzo[1,3]dioxol-5-yl, pyridin-3-yl, pyridin-4-yl, 2-benzonitrile, 1-phenyl-4-trifluoromethyl-1H-pyrazolyl, 4-bromophenyl, 2-benzoic acid methyl ester, N-3-phenyl-acetamide, 3-chloro-4-fluoro-phenyl, 2,3-dimethyl-phenyl, 2,6-difluoro-3-methyl-phenyl, 4-tert-butyl-phenyl, 4-dimethylamino-phenyl, 4-methoxy-3-trifluoromethyl-phenyl, 2-methyl-3-trifluoromethyl-phenyl, 2-amino-phenyl, 5-(4-methanesulfonyl-phenyl)-furan-2-yl, 2-difluoromethyl sulfanyl-phenyl, N,N-diethyl-4-benzenesulfonamide, 2-phenoxy-phenyl, 2,4,6-trimethyl-phenyl, 5-chloro-2-trifluoromethyl-phenyl, 2-fluoro-4-trifluoromethyl-phenyl, 2-fluoro-4-methoxy-phenyl, 4-benzoic acid, 2,2-difluoro-benzo[1,3]dioxol-5-yl, benzoic acid 2-benzyl ester.

38. The method according to claim 1, wherein said compound has formula (X): 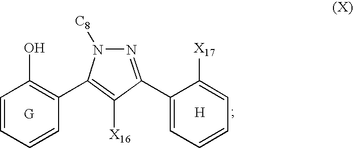
or a pharmaceutically acceptable salt thereof;
wherein:
C₈ is selected from H, aryl, heterocyclic, heteroaryl, aliphatic, C(O)R², C(O)R³, C(O)NH₂, C(O)NHR², C(O)NHR³, C(O)N(R²)₂, C(O)N(R³)₂;
X₁₆ is selected from selected from (CH₂)ₙ—Y, R², R³, R⁴, R⁵ or R⁶;
X₁₇ is CN, tetrazolyl, SO₂R², SO₂R³, SO₂NHR², SO₂NHR³, SO₂NR²R³, SO₂N(R²)₂;
wherein each of ring G, optionally including the hydroxyl group, C₈, and ring H optionally comprises up to 4 substituents independently selected from R¹, R², R³, R⁴, or R⁵;
R¹ is oxo, R⁶ or (CH₂)ₙ—Y;
n is 0, 1 or 2;
Y is halo, CN, NO₂, CHF₂, CH₂F, CF₃, OCF₃, OH, SCHF₂, SR⁶, S(O)R⁶, SO₂R⁶, NH₂, NHR⁶, N(R⁶)₂, NR⁶R⁸, COOH, COOR⁶, or OR⁶; or
two R¹ on adjacent ring atoms, taken together, form 1,2-methylenedioxy, 1,2-difluoromethylenedioxy, or 1,2-ethylenedioxy;
R² is aliphatic, wherein each R² optionally comprises up to 2 substituents independently selected from R¹, R⁴, or R⁵;
R³ is a cycloaliphatic, aryl, heterocyclic, or heteroaryl ring optionally comprising up to 3 substituents, independently selected from R¹, R², R⁴ or R⁵;
R⁴ is OR⁵, OR⁶, OC(O)R⁶, OC(O)R⁵, OC(O)OR⁶, OC(O)OR⁵, OC(O)N(R⁶)₂, OC(O)N(R⁵)₂, OC(O)N(R⁶R⁵), OP(O)(OR⁶)₂, OP(O)(OR⁵)₂, OP(O)(OR⁶)(OR⁵), SR⁶, SR⁵, S(O)R⁶, S(O)R⁵, SO₂R⁶, SO₂R⁵, SO₂N(R⁶)₂, SO₂N(R⁵)₂, SO₂NR⁵R⁶, SO₃R⁶, SO₃R⁵, C(O)R⁵, C(O)OR⁵, C(O)R⁶, C(O)OR⁶, C(O)N(R⁶)₂, C(O)N(R⁵)₂, C(O)N(R⁵R⁶), C(O)N(OR⁶)R⁶, C(O)N(OR⁵)R⁶, C(O)N(OR⁶)R⁵, C(O)N(OR⁵)R⁵, C(NOR⁶)R⁶, C(NOR⁶)R⁵, C(NOR⁵)R⁶, C(NOR⁵)R⁵, N(R⁶)₂, N(R⁵)₂, N(R⁵R⁶)₅, NR⁵C(O)R⁵, NR⁶C(O)R⁶, NR⁶C(O)R⁵, NR⁶C(O)OR⁶, NR⁵C(O)OR⁶, NR⁶C(O)OR⁵, NR⁵C(O)OR⁵, NR⁶C(O)N(R⁶)₂, NR⁶C(O)NR⁵R⁶, NR⁶C(O)N(R⁵)₂, NR⁵C(O)N(R⁶)₂, NR⁵C(O)NR⁵R⁶, NR⁵C(O)N(R⁵)₂, NR⁶SO₂R⁶, NR⁶SO₂R⁵, NR⁵SO₂R⁵, NR⁶SO₂N(R⁶)₂, NR⁶SO₂NR⁵R⁶, NR⁶SO₂N(R⁵)₂, NR⁵SO₂NR⁵R⁶, NR⁵SO₂N(R⁵)₂, N(OR⁶)R⁶, N(OR⁶)R⁵, N(OR⁵)R⁵, N(OR⁵)R⁶, P(O)(OR⁶)N(R⁶)₂, P(O)(OR⁶)N(R⁵R⁶), P(O)(OR⁶)N(R⁵)₂, P(O)(OR⁵)N(R⁵R⁶), P(O)(OR⁵)N(R⁶)₂, P(O)(OR⁵)N(R⁵)₂, P(O)(OR⁶)₂, P(O)(OR⁵)₂, or P(O)(OR⁶)(OR⁵);
R⁵ is a cycloaliphatic, aryl, heterocyclic, or heteroaryl ring optionally comprising up to 3 R¹ substituents;
R⁶ is H or aliphatic, wherein R⁶ optionally comprises a R⁷ substituent;
R⁷ is a cycloaliphatic, aryl, heterocyclic, or heteroaryl ring and each R⁷ optionally comprising up to 2 substituents independently chosen from H, (C₁-C₆)-straight or branched alkyl, (C₂-C₆) straight or branched alkenyl or alkynyl, 1,2-methylenedioxy, 1,2-ethylenedioxy, or (CH₂)ₙ—Z;
Z is selected from halo, CN, NO₂, CF₃, OCF₃, OH, S-aliphatic, S(O)-aliphatic, SO₂-aliphatic, NH₂, N-aliphatic, N(aliphatic)₂, N(aliphatic)R⁸, COOH, C(O)O(-aliphatic, or O-aliphatic; and
R⁸ is an amino protecting group.

39. The method according to claim 38, wherein X₁₆ and C₈ are H.

40. The method according to claim 39, wherein X₁₇ is CN, SO₂[(C1-C6)aliphatic], SO₂(phenyl), SO₂NH[(C1-C6)aliphatic], or SO₂NH(phenyl).

41. The method according to claim 1, wherein said ABC-transporter or a fragment thereof is in vivo.

42. The method according to claim 1, wherein said ABC-transporter or a fragment thereof is in vitro.

43. The method according to claim 41 or 42, wherein said ABC-transporter is CFTR.

44. A method of treating an ABC transporter mediated disease in a mammal, comprising the step of administering to said mammal a composition comprising the step of administering to said mammal a composition comprising a compound according to any one of claims 1-40.

45. The method according to claim 44, wherein said disease is selected from immunodeficiency disorder, inflammatory disease, allergic disease, autoimmune disease, destructive bone disorder, proliferative disorder, infectious disease or viral disease.

46. The method according to claim 45, wherein said disease is selected from Tangier's disease, stargardt disease 1, age related macular dystrophy 2, retinintis pigmentosa, dry eye disease, bare lymphocyte syndrome, PFIC-3, anemia, progressive intrahepatic cholestasis-2, Dublin-Johnson syndrome, Pseudoxanthoma elasticum, cystic fibrosis, familial persistent hyperinsulinemic hyproglycemia of infancy, adrenolecukodystrophy, sitosterolemia, chronic obstructive pulmonary disease, asthma, disseminated bronchiectasis, chronic pancreatitis, male infertility, emphysema, or pneumonia.

47. The method according to claim 46, wherein said disease is cystic fibrosis.

48. The method according to claim 45, wherein said disease is secretory diarrhea or polycystic kidney disease in a mammal.

49-84. (canceled)
